(19) United States
(12) Patent Application Publication
(10) Pub. No.: US 2022/0400964 A1
(43) Pub. Date: Dec. 22, 2022

(54) MEASUREMENT APPARATUS AND METHOD FOR CONTROLLING MEASUREMENT APPARATUS

(71) Applicant: Panasonic Intellectual Property Management Co., Ltd., Osaka (JP)

(72) Inventors: SHUNSUKE IMAI, Osaka (JP); TAKAMASA ANDO, Osaka (JP); HITOSHI TOMINAGA, Kyoto (JP)

(21) Appl. No.: 17/820,266

(22) Filed: Aug. 17, 2022

Related U.S. Application Data
(63) Continuation of application No. PCT/JP2021/005395, filed on Feb. 15, 2021.

(30) Foreign Application Priority Data
Mar. 9, 2020 (JP) 2020-039728
Jan. 28, 2021 (JP) 2021-012027

(51) Int. Cl. A61B 5/026; G01N 21/27; A61B 5/00

(52) U.S. Cl. CPC A61B 5/0261; G01N 21/27; A61B 5/0042; A61B 2576/026

(57) ABSTRACT

A measurement apparatus includes a light source, a sensor including light detection cells including a first light detection cell and a second light detection cell, and an electronic circuit. The circuit causes the light source to emit a light pulse, causes the first light detection cell to detect a reflected light pulse from a target in an exposure period including at least part of a period from when an intensity of the reflected light pulse starts increasing to when it starts falling and generate a first signal, causes the second light detection cell to detect the reflected light pulse in an exposure period including at least part of a trailing period from when the intensity of the reflected light pulse starts falling to when it stops falling and generate a second signal. The circuit generates, based on the first and second signals, data representing states of the target.

### BACKGROUND

### 1. Technical Field

The present disclosure relates to a measurement apparatus and a method for controlling a measurement apparatus.

### 2. Description of the Related Art

Various methods for measuring a biological signal caused by the brain activity of a subject have been developed. For example, Japanese Unexamined Patent Application Publication No. 2017-009584 discloses an example of an image pickup apparatus that acquires, in a state where the image pickup apparatus does not come in contact with a target, information representing changes over time in the cerebral blood flow of a subject.

### SUMMARY

One non-limiting and exemplary embodiment provides a technology that enables acquisition of information regarding a shallow site and a deep site of a measurement target with higher temporal resolution than before.

In one general aspect, the techniques disclosed here feature a measurement apparatus including a light source that emits a first light pulse toward a target, a sensor including light detection cells including a first light detection cell and a second light detection cell, and an electronic circuit that controls the light source and the sensor and processes a signal output from the sensor. The electronic circuit causes the light source to emit a first light pulse, causes the first light detection cell to detect a first reflected light pulse in a first exposure period and generate, based on the first reflected light pulse detected in the first exposure period, a first signal, the first reflected light pulse being generated based on the first light pulse and coming from the target, the first exposure period including at least part of a period from when an intensity of the first reflected light pulse starts increasing to when the intensity of the first reflected light pulse starts falling, causes the second light detection cell to detect the first reflected light pulse in a second exposure period and generate, based on the first reflected light pulse detected in the second exposure period, a second signal, the second exposure period including at least part of a trailing period from when the intensity of the first reflected light pulse starts falling to when the intensity of the first reflected light pulse stops falling, generates, based on the first signal, and outputs first data representing a state of a surface of the target, and generates, based on the second signal, and outputs second data representing a state inside the target.

A general or specific embodiment according to the present disclosure may be realized by a system, an apparatus, a method, an integrated circuit, a computer program, or a computer readable recording medium such as a recording disc or by a combination of some or all of a system, an apparatus, a method, an integrated circuit, a computer program, and a recording medium. Examples of the computer readable recording medium may include a nonvolatile recording medium such as a compact disc read-only memory (CD-ROM). The apparatus may be formed by one or more devices. In a case where the apparatus is formed by two or more devices, the two or more devices may be arranged in one apparatus or may be arranged in two or more separate apparatuses in a divided manner.

In the present specification and the claims, an “apparatus” may refer not only to one apparatus but also to a system formed by apparatuses.

With a measurement apparatus according to the present disclosure, information regarding a shallow site and a deep site of a measurement target can be acquired with higher temporal resolution than before.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic diagram illustrating a measurement apparatus according to a first embodiment;

FIG. 2A is a diagram illustrating an example in which a light emission pulse has an impulse waveform;

FIG. 2B is a diagram illustrating an example in which a light emission pulse has a rectangular waveform;

FIG. 3 is a diagram illustrating an example of the configuration of an image sensor;

FIG. 4A is a diagram illustrating an example of an operation within one frame;

FIG. 4B is a diagram illustrating another example of a detection operation using light having two wavelengths;

FIG. 5 is a schematic diagram for describing relationships between timings of a light emission pulse and a reflected light pulse and individual exposure periods;

FIG. 6A is a diagram illustrating an example of an arrangement pattern of two kinds of pixels of the image sensor, the two kinds of pixels having different exposure periods;

FIG. 6B is a diagram illustrating another example of the arrangement pattern of the two kinds of pixels of the image sensor, the two kinds of pixels having different exposure periods;

FIG. 6C is a diagram illustrating yet another example of the arrangement pattern of the two kinds of pixels of the image sensor, the two kinds of pixels having different exposure periods;

FIG. 6D is a diagram illustrating yet another example of the arrangement pattern of the two kinds of pixels of the image sensor, the two kinds of pixels having different exposure periods;

FIG. 7 is a flow chart illustrating the summary of an operation in the first embodiment;

FIG. 8 is a schematic diagram for describing relationships between timings of a light emission pulse and a reflected light pulse according to the first embodiment and individual exposure periods;

FIG. 9A is a diagram illustrating an example of an arrangement pattern of three kinds of pixels of the image sensor, the three kinds of pixels having different exposure periods;

FIG. 9B is a diagram illustrating another example of the arrangement pattern of the three kinds of pixels of the image sensor, the three kinds of pixels having different exposure periods;

FIG. 9C is a diagram illustrating yet another example of the arrangement pattern of the three kinds of pixels of the image sensor, the three kinds of pixels having different exposure periods;

FIG. 9D is a diagram illustrating yet another example of the arrangement pattern of the three kinds of pixels of the image sensor, the three kinds of pixels having different exposure periods;

FIG. 10 is a flow chart illustrating the summary of an operation in a second embodiment;

FIG. 11 is a timing chart illustrating a modification of the second embodiment;

FIG. 12 is a timing chart illustrating another modification of the second embodiment;

FIG. 13 is a diagram for describing the principle of a distance calculation;

FIG. 14 is a diagram illustrating yet another modification;

FIG. 15 is a diagram illustrating examples of changes over time in three kinds of generated images;

FIG. 16 is a diagram illustrating an example of a distance image;

FIG. 17 is a diagram schematically illustrating the procedure of an operation for generating a correction value image to be used in correction processing;

FIG. 18 is a diagram schematically illustrating correction processing using the correction value image; and

FIG. 19 is a diagram illustrating a measurement operation according to yet another modification.

### DETAILED DESCRIPTIONS

Any one of embodiments to be described below is intended to represent a general or specific example. Numerical values, shapes, materials, constituent elements, arrangement positions and connection forms of the constituent elements, steps, and the order of steps are examples, and are not intended to limit the technologies of the present disclosure. Among the constituent elements of the following embodiments, constituent elements that are not described in independent claims representing the most generic concept will be described as optional constituent elements. Each drawing is a schematic diagram and is not necessarily precisely illustrated. Furthermore, in each drawing, substantially the same or similar constituent elements are denoted by the same reference signs. Redundant description may be omitted or simplified.

In the present disclosure, all or some of circuits, units, devices, members, or portions or all or some of the functional blocks of a block diagram may be executed by, for example, one or more electronic circuits including a semiconductor device, a semiconductor integrated circuit (IC), or a large-scale integration circuit (LSI). The LSI or the IC may be integrated onto one chip or may be formed by combining chips. For example, functional blocks other than a storage device may be integrated onto one chip. In this case, the term LSI or IC is used; however, the term(s) to be used may change depending on the degree of integration, and the terms system LSI, very large-scale integration circuit (VLSI), or ultra-large-scale integration circuit (VLSI) may be used. A field-programmable gate array (FPGA) or a reconfigurable logic device that allows reconfiguration of interconnection inside the LSI or setup of a circuit section inside the LSI can also be used for the same purpose, the FPGA and the reconfigurable logic device being programmed after the LSIs are manufactured.

Furthermore, functions or operations of all or some of the circuits, the units, the devices, the members, or the portions can be executed through software processing. In this case, software is recorded in one or more non-transitory recording mediums such as a read-only memory (ROM), an optical disc, or a hard disk drive, and when the software is executed by a processing device (a processor), the function specified by the software is executed by the processing device (the processor) and peripheral devices. The system or the apparatus may have the one or more non-transitory recording mediums in which the software is recorded, the processing device (the processor), and a hardware device to be needed such as an interface.

First, the summary of embodiments of the present disclosure will be described.

A measurement apparatus according to an embodiment, which is an example, of the present disclosure includes a light source that emits a first light pulse toward a target, a sensor including light detection cells including a first light detection cell and a second light detection cell, and an electronic circuit that controls the light source and the sensor and processes a signal output from the sensor. The electronic circuit performs operations (a) to (e) below.

(a) The electronic circuit causes the light source to emit a first light pulse.
(b) The electronic circuit causes the first light detection cell to detect a first reflected light pulse in a first exposure period and generate, based on the first reflected light pulse detected in the first exposure period, a first signal, the first reflected light pulse being generated based on the first light pulse and coming from the target, the first exposure period including at least part of a period from when an intensity of the first reflected light pulse starts increasing to when the intensity of the first reflected light pulse starts falling.
(c) The electronic circuit causes the second light detection cell to detect the first reflected light pulse in a second exposure period and generate, based on the first reflected light pulse detected in the second exposure period, a second signal, the second exposure period including at least part of a trailing period from when the intensity of the first reflected light pulse starts falling to when the intensity of the first reflected light pulse stops falling.
(d) The electronic circuit generates, based on the first signal, and outputs first data representing a state of a surface of the target.
(e) The electronic circuit generates, based on the second signal, and outputs second data representing a state inside the target.

In this case, the “target” may be, for example, a living body such as the head of a person. A light pulse from the light source may be emitted toward, for example, the forehead of the person. When the light pulse is incident on the forehead, the light pulse is reflected or scattered by the surface or inside of the forehead, so that a reflected light pulse occurs. The reflected light pulse includes a surface reflection component, which is reflected at the surface of the target, and an internal scattering component, which is scattered inside the target. The sensor receives the reflected light pulse using the first light detection cell in the first exposure period and generates the first signal based on the amount of light received. The sensor also receives the reflected light pulse using the second light detection cell in the second exposure period and generates the second signal based on the amount of light received. The first signal reflects the intensity of a light component that has been reflected or scattered at or near the surface of the target and has returned. The second signal reflects the intensity of a light component that has been scattered by internal tissue of a deeper site of the target and has returned. The electronic circuit generates, based on the first signal, first data reflecting the state of the surface of the target. In the following description, the first data may also be referred to as “surface layer data”. The electronic circuit also generates, based on the second signal, second data reflecting a state inside the target. In the following description, the second data may also be referred to as “deep-site data”. The electronic circuit may output, as the first data, the first signal as is or may output, as the first data, data newly generated through a calculation using the first signal. Similarly, the electronic circuit may output, as the second data, the second signal as is or may output, as the second data, a signal newly generated through a calculation using the second signal. In a case where a target site to be measured is the forehead of a person, the second data depends on, for example, the state of the brain activity of the person.

With the above-described configuration, the sensor can detect, out of the reflected light pulse, a component that returns relatively early by using the first light detection cell and a component that returns relatively late by using the second light detection cell. Thus, compared with a case where these two components are detected by using one light detection cell, the temporal resolution can be increased.

The second exposure period may start after the trailing period of the first reflected light pulse starts. With such a configuration, information regarding a deeper site of the target can be acquired with higher accuracy.

The first exposure period may include at least part of a rising period from when the intensity of the first reflected light pulse starts increasing to when the intensity of the first reflected light pulse stops increasing. With such a configuration, information regarding a site closer to the surface layer of the target can be acquired with higher accuracy.

The first exposure period may end before the trailing period starts. With such a configuration, the overlap between the first exposure period and the second exposure period can be reduced, and thus information regarding the surface layer and information regarding the deep site can be acquired with higher accuracy.

In the present specification, the “rising period” of a light pulse refers to the period from when the intensity of the light pulse starts increasing to when the intensity of the light pulse stops increasing at the position of a light reception surface of the sensor. More strictly speaking, the “rising period” is defined as the period from when the intensity of the light pulse exceeds a preset lower limit value to when the intensity of the light pulse reaches a preset upper limit value. The lower limit value may be set to, for example, a value corresponding to 10% of a peak value of the intensity of the light pulse, and the upper limit value may be set to, for example, a value corresponding to 90% of the peak value. In contrast, the “trailing period” of a light pulse refers to the period from when the intensity of the light pulse starts falling to when the intensity of the light pulse stops falling at the position of the light reception surface of the sensor. More strictly speaking, the “trailing period” refers to the period from when the intensity of the light pulse falls below a preset upper limit value to when the intensity of the light pulse reaches a preset lower limit value. Regarding the trailing period, the upper limit value may also be set to, for example, the value corresponding to 90% of the peak value of the intensity of the light pulse, and the lower limit value may also be set to, for example, the value corresponding to 10% of the peak value.

The light detection cells may include first light detection cells including the first light detection cell and second light detection cells including the second light detection cell. In that case, the electronic circuit may perform the following operations.

(b1) The electronic circuit causes each of the first light detection cells to detect the first reflected light pulse in the first exposure period and generate, based on the first reflected light pulse detected in the first exposure period, the first signal.
(c1) The electronic circuit causes each of the second light detection cells to detect the first reflected light pulse in the second exposure period and generate, based on the first reflected light pulse detected in the second exposure period, the second signal.
(d1) The electronic circuit generates, based on first signals including the first signal, and outputs the first data, the first signals being output from the respective first light detection cells.
(e1) The electronic circuit generates, based on second signals including the second signal, and outputs the second data, the second signals being output from the respective second light detection cells.

With the above-described configuration, information regarding the surface layer of the target and information regarding a deep site of the target can be acquired over a wider range.

The number of first light detection cells may be smaller than, greater than, or equal to the number of second light detection cells.

The sensor may be an image sensor. The light detection cells may be arranged in a matrix. The electronic circuit may generate, as the first data, image data based on the first signals, and generate, as the second data, image data based on the second signals. With such a configuration, image data representing the state of the surface layer of the target and image data representing the state of the inside of the target can be output. By displaying an image based on these pieces of image data on a display, the state of the surface layer and the inside of the target can be visualized.

Regarding the light detection cells arranged in a matrix, there may be various arrangement patterns. For example, rows or columns formed by the first light detection cells and rows or columns formed by the second light detection cells may be aligned in an alternating manner. Alternatively, the first light detection cells and the second light detection cells may be arranged in a checkerboard pattern.

In a case where the target includes a person's head, the first data may represent an appearance of a face of the head. Moreover, the second data may represent a cerebral blood flow state of the head. With such a configuration, an image of the face and an image representing the state of the cerebral blood flow can be generated and displayed.

The light detection cells may further include a third light detection cell. The electronic circuit may cause the first light detection cell or the third light detection cell to detect the first reflected light pulse and generate a third signal in a third exposure period, the third exposure period being different from the first exposure period and the second exposure period. The electronic circuit may generate and output third data based on the third signal. The third data may represent, for example, a state of a scalp blood flow. With such a configuration, for example, three kinds of data can be generated, which are the first data representing the facial appearance, the second data representing the cerebral blood flow, and the third data representing the scalp blood flow.

The electronic circuit may cause the first light detection cell to generate the third signal in the third exposure period, and generate, based on the first signal and the third signal, and output data representing a distance from the sensor to the target. With such a configuration, not only information regarding the surface layer of the target and information regarding the inside of the target but also information regarding the distance can be acquired.

The light source may further emit a second light pulse toward the target. The electronic circuit may further perform the following operations.

(a2) The electronic circuit causes the light source to emit the second light pulse after the electronic circuit causes the light source to emit the first light pulse.
(b2) The electronic circuit causes the first light detection cell to detect a second reflected light pulse in a third exposure period and generate, based on the second reflected light pulse detected in the third exposure period, a third signal, the second reflected light being generated based on the second light pulse and coming from the target, the third exposure period including at least part of a trailing period from when an intensity of the second reflected light pulse starts falling to when the intensity of the second reflected light pulse stops falling. In this case, the length from the time at which emission of the first light pulse is started to the time at which the first exposure period starts may be different from the length from the time at which emission of the second light pulse is started to the time at which the third exposure period starts.
(c2) The electronic circuit causes the second light detection cell to detect the second reflected light pulse in a fourth exposure period and generate, based on the second reflected light pulse detected in the fourth exposure period, a fourth signal, the fourth exposure period being different from the third exposure period and including at least part of the trailing period of the second reflected light pulse.
(d2) The electronic circuit generates, based on the first signal and the third signal, and outputs data representing a distance from the sensor to the target.

In this case, the data representing the distance may be generated as the “first data” described above or as data independent of the “first data” described above.

In a case where the target includes a person's head, the electronic circuit may generate, based on the first signal and the second signal, and outputs data representing the person's psychological state or body condition. The data may represent, for example, the state of the person's interest, emotions, sleepiness, level of concentration, or fatigue. For example, by combining information regarding the person's facial expression represented by the first signal and information regarding the person's brain activity represented by the second signal, the person's psychological state or body condition can be estimated.

In a case where the sensor generates the third signal and the fourth signal, the electronic circuit may further use the third signal and the fourth signal in addition to the first signal and the second signal to generate data representing the person's psychological state or body condition. For example, in a case where the third signal represents the state of the scalp blood flow of the person, the psychological state or body condition can be estimated with higher accuracy by using information representing the state of the scalp blood flow in addition to information representing the facial expression and the cerebral blood flow.

The light source may further emit a second light pulse toward the target. A wavelength of the second light pulse may differ from a wavelength of the first light pulse. The first exposure period may start when a first time elapses from a time at which emission of the first light pulse is started. The electronic circuit may further perform the following operations.

(a3) The electronic circuit causes the light source to emit the first light pulse and the second light pulse in each of the first measurement period and the second measurement period.
(b3) The electronic circuit causes the first light detection cell to detect the first reflected light pulse in the first exposure period included in the first measurement period and a second reflected light pulse generated based on the second light pulse and coming from the target in a third exposure period, which starts when a second time elapses from a time at which emission of the second light pulse is started in the first measurement period, and generate the first signal.
(c3) The electronic circuit causes the first light detection cell to detect the first reflected light pulse in a fourth exposure period, which starts when a third time elapses from a time at which emission of the first light pulse is started in the second measurement period, and the second reflected light pulse in a fifth exposure period, which starts when a fourth time elapses from a time at which emission of the second light pulse is started in the second measurement period, and generate a third signal.
(d3) The electronic circuit generates, based on the first signal and the third signal, data representing a distance from the sensor to the target.

In this example, the third time may differ from the first time, and the fourth time may differ from the second time.

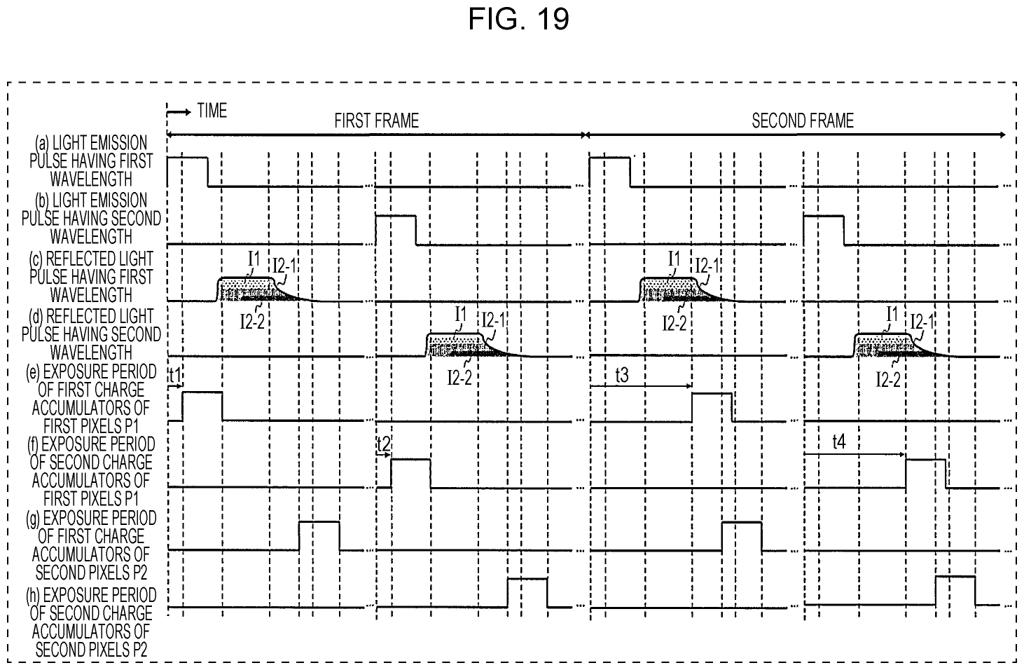

By performing the above-described operations, as will be described with reference to FIG. 19, data representing the distance from the sensor to the target can be generated every two continuous measurement periods. In a case where the sensor is an image sensor, the electronic circuit may repeatedly generate a distance image based on the first signal and the third signal and an image based on the second signal. In that case, the first measurement period and the second measurement period may also be referred to as a first frame period and a second frame period, respectively.

The electronic circuit may perform the following operations.

(a4) The electronic circuit causes the light source to emit one or more light pulses.
(b4) The electronic circuit causes the first light detection cell to detect a first component of one or more reflected light pulses and generate a first signal, the one or more reflected light pulses being generated based on the one or more light pulses and coming from a living body.
(c4) The electronic circuit causes the second light detection cell to detect a second component of the one or more reflected light pulses and generate a second signal.
(d4) The electronic circuit causes the first light detection cell to detect a third component of the one or more reflected light pulses and generate a third signal.
(e4) The electronic circuit generates, based on the first signal and the third signal, first data representing a distance from the sensor to the living body.
(f4) The electronic circuit generates, based on the second signal, second data representing a blood flow state of the living body.

By performing such operations, the first data representing the distance and the second data representing the state of the blood flow of the living body can be generated with high resolution.

The electronic circuit may correct, based on the first data, the second data. By performing such a correction, even in a case where the living body has moved during a measurement, blood flow information regarding the living body can be acquired with high accuracy.

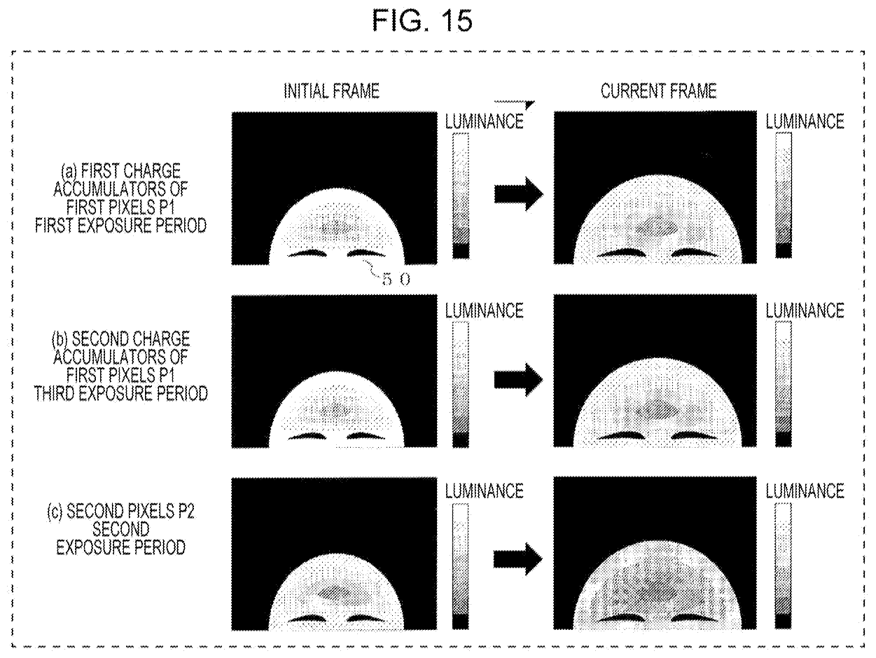

The electronic circuit may correct, based on data representing a spatial distribution of illuminance of the one or more light pulses and the first data, the second data. The data representing the spatial distribution of illuminance may be prepared in advance before measurement and be stored in a storage medium. Details of a method for correcting the second data representing the blood flow state of the living body on the basis of the first data representing the distance from the sensor to the target and the data representing the spatial distribution of illuminance will be described later with reference to FIGS. 15 to 18.

A method according to an aspect of the present disclosure is a method for controlling a measurement apparatus including a light source and a sensor including light detection cells including a first light detection cell and a second light detection cell, the method including: causing the light source to emit a light pulse, causing the first light detection cell to detect a reflected light pulse in a first exposure period and generate, based on the reflected light pulse detected in the first exposure period, a first signal, the reflected light pulse being generated based on the light pulse and coming from a target, the first exposure period including at least part of a period from when an intensity of the reflected light pulse starts increasing to when the intensity of the reflected light pulse starts falling, causing the second light detection cell to detect the reflected light pulse in a second exposure period and generate, based on the reflected light pulse detected in the second exposure period, a second signal, the second exposure period including at least part of a trailing period from when the intensity of the reflected light pulse starts falling to when the intensity of the reflected light pulse stops falling, generating, based on the first signal, and outputting first data representing a state of a surface of the target, and generating, based on the second signal, and outputting second data representing a state inside the target.

A method according to another aspect of the present disclosure is a method for controlling a measurement apparatus including a light source and a sensor including light detection cells including a first light detection cell and a second light detection cell, the method including: causing the light source to emit one or more light pulses, causing the first light detection cell to detect a first component of one or more reflected light pulses and generate a first signal, the one or more reflected light pulses being generated based on the one or more light pulses and coming from a living body, causing the second light detection cell to detect a second component of the one or more reflected light pulses and generate a second signal, causing the first light detection cell to detect a third component of the one or more reflected light pulses and generate a third signal, generating, based on the first signal and the third signal, first data representing a distance from the sensor to the living body, and generating, based on the second signal, second data representing a blood flow state of the living body.

An apparatus according to another embodiment of the present disclosure includes one or more processors and a storage medium in which a computer program to be executed by the one or more processors is stored. The one or more processors may execute the functions of the electronic circuit in any one of the examples described above by executing the computer program.

The present disclosure includes a computer program that defines the functions of the above-described electronic circuit and a control method that the above-described electronic circuit executes.

In the following, embodiments of the present disclosure, which are examples, will be more specifically described with reference to the drawings.

### First Embodiment

### [1. Measurement Apparatus 100]

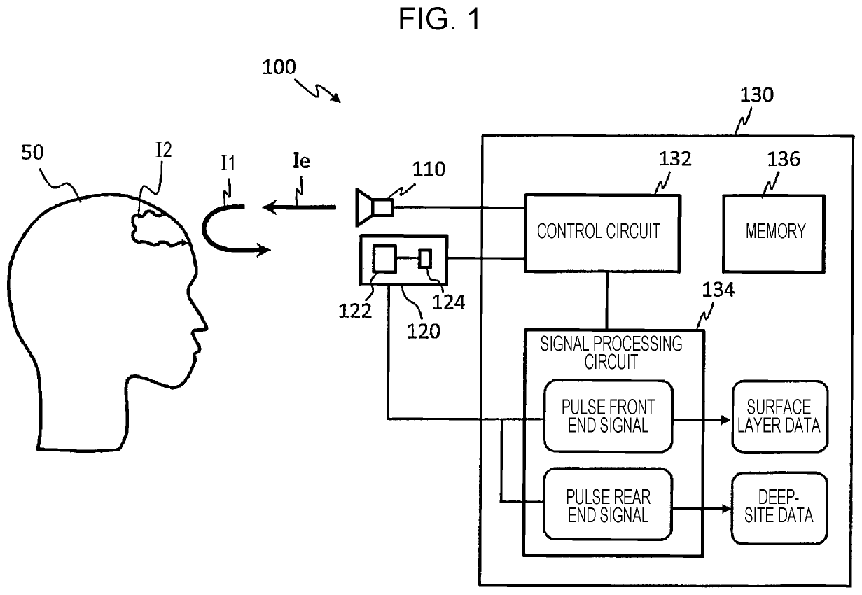

FIG. 1 is a schematic diagram illustrating a measurement apparatus 100 according to a first embodiment, which is an example, of the present disclosure. The measurement apparatus 100 has a light source 110, an image sensor 120, and an electronic circuit 130. The image sensor 120 has light detection cells. Each light detection cell includes a photoelectric converter 122 and one or more charge accumulators 124. The electronic circuit 130 includes a control circuit 132, a signal processing circuit 134, and a storage medium such as a memory 136.

FIG. 1 also illustrates the head of a subject 50 serving as a target to be measured by the measurement apparatus 100. Note that targets are not limited to the heads of persons. Targets may be other kinds of living body such as animals or plants other than persons. Depending on a use, a target may be an object other than living bodies. For example, a scatterer such as liquid, gas, or food may be treated as a target.

The measurement apparatus 100 according to the present embodiment can acquire, in a contactless manner, information representing the state of the scalp blood flow and cerebral blood flow of the subject 50 to be observed. The measurement apparatus 100 may generate, for example, data of a two-dimensional image representing the concentration distribution of at least one of oxygenated hemoglobin or deoxygenated hemoglobin in the brain of the subject 50. Alternatively, the measurement apparatus 100 may generate other kinds of data that change due to the brain activity of the subject 50.

The light detection cells of the image sensor 120 include a first light detection cell and a second light detection cell. In the following description, the light detection cells may be referred to as “pixels”, the first light detection cell may be referred to as a “first pixel P1”, and the second light detection cell may be referred to as a “second pixel P2”. The image sensor 120 can acquire, for example, information regarding the facial appearance or the scalp blood flow of the subject 50 using the first light detection cell and information regarding the cerebral blood flow of the subject 50 using the second light detection cell. The signal processing circuit 134 can execute, with high temporal resolution, processing for generating surface layer data representing the state of the facial appearance or scalp blood flow of the subject 50 and deep-site data representing the state of the cerebral blood flow of the subject 50.

In the following, details of the individual constituent elements will be described.

### [1-1. Light Source 110]

The light source 110 is arranged so as to emit light toward the head of the subject 50, such as a target area including the forehead of the subject 50. Light that has been emitted from the light source 110 and reached the subject 50 is divided into a surface reflection component I1, which is reflected at the surface of the subject 50, and an internal scattering component I2, which is scattered inside the subject 50. The internal scattering component I2 is a component reflected or scattered one time or scattered multiple times inside the living body. In a case where light is emitted toward the forehead of a person as in the present embodiment, the internal scattering component I2 refers to a component that reaches a site that is about 8 mm to 16 mm deep from the surface of the forehead such as the brain of the person and returns to the measurement apparatus 100 again. The surface reflection component I1 includes three components, which are a direct reflection component, a diffusion reflection component, and a scattering reflection component. The direct reflection component is a reflection component whose incidence and reflection angles are equal to each other. The diffusion reflection component is a component that is scattered and reflected by surface roughness. The scattering reflection component is a component that is scattered and reflected by internal tissue near the surface. In a case where light is emitted toward the head of a person, the scattering reflection component is a component that is scattered and reflected inside the outer layer of the skin. The surface reflection component I1 may include these three components. The directions in which the surface reflection component I1 and the internal scattering component I2 travel are changed by reflection or scattering, and part of the surface reflection component I1 and the internal scattering component I2 reaches the image sensor 120. The surface reflection component I1 includes surface information regarding the subject 50, such as blood flow information regarding the face and scalp. The internal scattering component I2 includes internal information regarding the subject 50, such as cerebral blood flow information.

In the present embodiment, the surface reflection component I1 and the internal scattering component I2 of reflected light returning from the head of the subject 50 are detected. The surface reflection component I1 reflects the state of the facial appearance or scalp blood flow of the subject 50. Thus, changes in the state of the facial appearance or scalp blood flow of the subject 50 can be estimated by analyzing changes over time in the surface reflection component I1. In contrast, the intensity of the internal scattering component I2 changes, reflecting the brain activity of the subject 50. Thus, the state of the brain activity of the subject 50 can be estimated by analyzing changes over time in the internal scattering component I2.

A method for acquiring the internal scattering component I2 will be described. The light source 110 emits a light pulse multiple times repeatedly at predetermined time intervals or predetermined timings in accordance with a command from the control circuit 132. The light pulse emitted from the light source 110 may be, for example, a rectangular wave for which the length of the trailing period is close to zero, the trailing period being from when the intensity of the light pulse starts falling to when the intensity of the light pulse stops falling. Generally, light incident on the head of the subject 50 propagates along various paths in the head and goes out from the surface of the head with time differences. Thus, the temporal rear end portion of the internal scattering component I2 of the light pulse has a temporal spread. In a case where the target area is the forehead of the subject 50, the width of the temporal rear end of the internal scattering component I2 is about 4 ns. When this is taken into consideration, the length of the trailing period, which is the period from when the intensity of the light pulse starts falling to when the intensity of the light pulse stops falling, may be set to, for example, less than or equal to 2 ns, which is less than or equal to half the temporal rear end of the internal scattering component I2. The trailing period may further be less than or equal to 1 ns, which is half the value. The length of the rising period of the light pulse emitted from the light source 110 can be set freely. To detect the internal scattering component I2 in the present embodiment, the trailing portion where the intensity of the light pulse is falling is used, and the rising portion where the intensity of the light pulse is increasing is not used. The rising portion of the light pulse is used to detect the surface reflection component I1.

The light source 110 may include, for example, a laser device such as a laser diode (LD). Light emitted from the laser device may be adjusted so as to have steep time response characteristics such that the trailing portion of the light pulse forms a substantially right angle with the time axis. The light source 110 may include a drive circuit that controls the driving current of the LD. The drive circuit may include, for example, an enhancement mode power transistor such as a field-effect transistor (GaN FET) including a gallium nitride (GaN) semiconductor. By using such a drive circuit, the trailing edge of the light pulse output from the LD can be made steep.

The wavelength of light emitted from the light source 110 may be, for example, a freely chosen wavelength included in a wavelength range greater than or equal to 650 nm and less than or equal to 950 nm. This wavelength range is included in the wavelength range from red to near infrared rays. The above-described wavelength range is called a “near-infrared window”, and light having this wavelength range is characterized by being relatively less likely to be absorbed by moisture in the living body and by the skin of the living body. In a case where a living body is a target to be detected, detection sensitivity can be increased by using light in the above-described wavelength range. As in the present embodiment, in a case where changes in the blood flow of the brain of a person are to be detected, it is conceivable that light used is absorbed mainly by oxygenated hemoglobin (HbO₂) and deoxygenated hemoglobin (Hb). Oxygenated hemoglobin differs from deoxygenated hemoglobin in wavelength dependence of light absorption. Generally, when a change occurs in blood flow, the concentrations of oxygenated hemoglobin and deoxygenated hemoglobin change. In response to this change, the degree of light absorbance changes. Thus, when a change occurs in blood flow, the amount of light detected changes temporally.

The light source 110 may emit light having a single wavelength included in the above-described wavelength range or may emit light having two or more wavelengths included in the above-described wavelength range. Light having wavelengths may be emitted from light sources in a respective manner.

Generally, the absorption characteristics and scattering characteristics of living tissue vary depending on the wavelength. Thus, the components of a target to be measured can be analyzed in more detail by detecting wavelength dependence of a light signal due to the internal scattering component I2. For example, in living tissue, when the wavelength is greater than or equal to 650 nm and less than 805 nm, a light absorption coefficient due to deoxygenated hemoglobin is larger than a light absorption coefficient due to oxygenated hemoglobin. When the wavelength is longer than 805 nm and less than or equal to 950 nm, the light absorption coefficient due to oxygenated hemoglobin is larger than the light absorption coefficient due to deoxygenated hemoglobin.

Thus, the light source 110 may be configured to emit light having a wavelength of greater than or equal to 650 nm and less than 805 nm (for example, about 750 nm) and light having a wavelength of longer than 805 nm and less than or equal to 950 nm (for example, about 850 nm). In this case, for example, the light intensity of the internal scattering component I2 due to light having a wavelength of about 750 nm and, for example, the light intensity of the internal scattering component I2 due to light having a wavelength of about 850 nm are to be measured. The light source 110 may also include a first light-emitting device that emits light having a wavelength of greater than or equal to 650 nm and less than 805 nm and a second light-emitting device that emits light having a wavelength of greater than 805 nm and less than or equal to 950 nm. The signal processing circuit 134 can obtain changes in the concentrations of HbO₂ and Hb in the blood from initial values by solving predetermined simultaneous equations on the basis of light intensity signal values input on a pixel basis.

The measurement apparatus 100 according to the present embodiment measures the cerebral blood flow of the subject 50 in a contactless manner. Thus, the light source 110 may be used, which is designed by taking effects on the retina into consideration. For example, the light source 110 may be used, which satisfies class 1 of laser safety standards developed in various countries. In a case where class 1 is satisfied, the subject 50 is irradiated with light of low illuminance for which the accessible emission limit (AEL) is lower than 1 mW. Note that the light source 110 itself does not have to satisfy class 1. For example, class 1 of the laser safety standards may be satisfied by arranging a diffusion plate or a neutral-density (ND) filter between the light source 110 and the subject 50 to diffuse or attenuate light.

Hitherto, a streak camera has been used to differentiate between pieces of information regarding, for example, absorption coefficients or scattering coefficients at different sites in the depth direction inside a living body and perform detection. For example, Japanese Unexamined Patent Application Publication No. H4-189349 discloses examples of such a streak camera. In these streak cameras, an ultrashort light pulse having a femtosecond or picosecond pulse width is used to perform measurement with a desired spatial resolution.

In contrast, the measurement apparatus 100 of the present embodiment can differentiate the surface reflection component I1 from the internal scattering component I2 and perform detection. Thus, the light pulse emitted by the light source 110 does not have to be an ultrashort light pulse and its pulse width can be freely selected.

In a case where the head of a person is irradiated with light in order to measure the cerebral blood flow, the light amount of the internal scattering component I2 may have a significantly small value that is about one several thousandths to one several ten thousandths the light amount of the surface reflection component I1. Furthermore, when the laser safety standards are taken into consideration, the amount of light with which irradiation can be performed is extremely low. Thus, it is extremely difficult to detect the internal scattering component I2. Even in that case, when the light source 110 emits a light pulse having a relatively large pulse width, the accumulated amount of internal scattering component I2 with a time lag can be increased. As a result, the amount of light to be detected can be increased, and a signal-to-noise (SN) ratio can be improved.

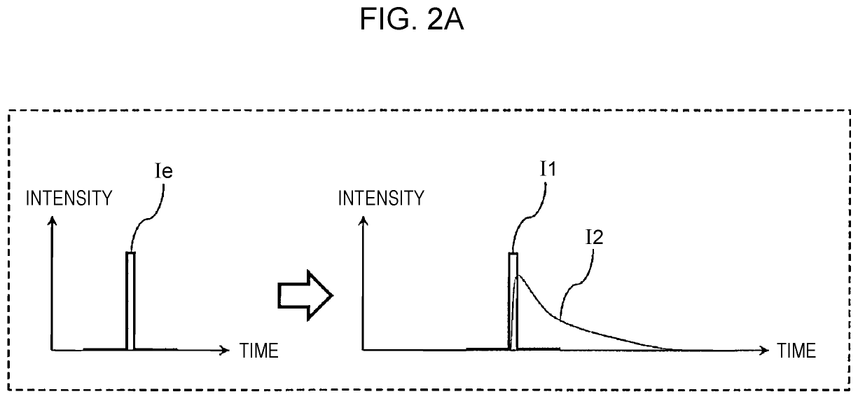

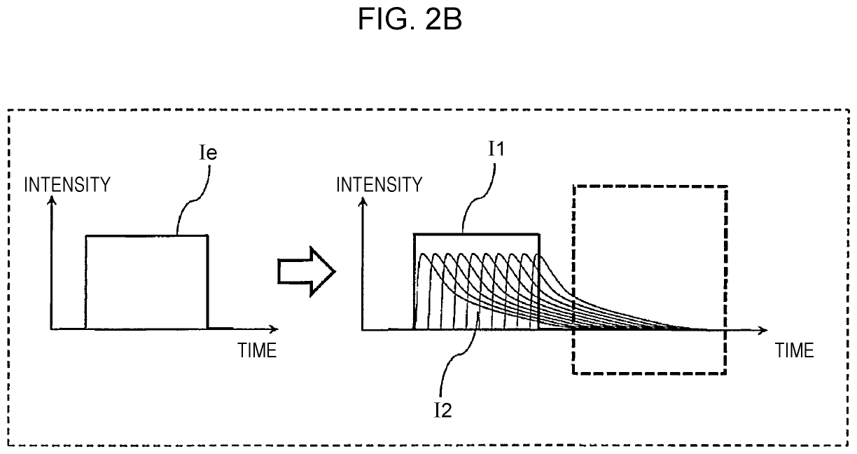

FIG. 2A and FIG. 2B are diagrams illustrating examples of changes over time in the intensity of a light emission pulse Ie and in the intensities of the surface reflection component I1 and the internal scattering component I2 of a reflected light pulse. FIG. 2A illustrates examples of respective waveforms in a case where the light emission pulse Ie has an impulse waveform. FIG. 2B illustrates examples of respective waveforms in a case where the light emission pulse Ie has a rectangular waveform. The internal scattering component I2 is actually very weak; however, in FIGS. 2A and 2B, the intensity of the internal scattering component I2 is illustrated in an exaggerated manner.

As illustrated in FIG. 2A, in a case where the light emission pulse Ie has an impulse waveform, the surface reflection component I1 has an impulse waveform similar to that of the light emission pulse Ie, and the internal scattering component I2 has an impulse response waveform delayed from that of the surface reflection component I1. This is because the internal scattering component I2 corresponds to a combination of light beams that have passed along various paths inside the skin.

As illustrated in FIG. 2B, in a case where the light emission pulse Ie has a rectangular waveform, the surface reflection component I1 has a rectangular waveform similar to that of the light emission pulse Ie, and the internal scattering component I2 has a waveform in which many impulse response waveforms are superposed with each other. The inventors confirmed that, compared with the case where the light emission pulse Ie has an impulse waveform, the light amount of the internal scattering component I2 to be detected by the image sensor 120 can be amplified by superimposing many impulse response waveforms. By starting opening the electronic shutter after a timing at which the intensity of the reflected light pulse starts falling, the internal scattering component I2 can be effectively detected. The broken line frame in the graph on the right side of FIG. 2B represents an example of a shutter open period during which the electronic shutter of the image sensor 120 is open. This shutter open period is also referred to as an “exposure period”. When the pulse width of the rectangular pulse is on the order of 1 ns to 10 ns, the light source 110 can be driven at a relatively low voltage, and the measurement apparatus 100 can be reduced in size and a reduction in cost can be achieved. In order to detect the internal scattering component I2 effectively, in the present embodiment, exposure is started after the timing at which the intensity of the surface reflection component I1 reaching the image sensor 120 starts falling.

The light source 110 may include, for example, a light-emitting device using a general-purpose semiconductor laser. In a case where a general-purpose semiconductor laser is driven at a low voltage, when the pulse width is too short, it becomes harder to follow driving for starting and stopping light emission. Thus, the light emission waveform varies from pulse light emission to pulse light emission, and unstable behaviors are more likely to be exhibited, so that distance measurement results are more likely to vary. In order to obtain a stable waveform by using a general-purpose semiconductor laser, the light source 110 may be controlled so that, for example, a light pulse having a pulse width of greater than or equal to 3 ns is emitted. Alternatively, in order to obtain a more stable waveform, the light source 110 may emit a light pulse having a pulse width of greater than or equal to 5 ns or even greater than or equal to 10 ns. In contrast, when the pulse width is too large, a light leak to the charge accumulators 124 at the time when the shutter is off, that is, parasitic light sensitivity (PLS) increases, so that a measurement error may be caused. Thus, the light source 110 may be controlled so as to generate, for example, a light pulse having a pulse width of less than or equal to 50 ns. Alternatively, the light source 110 may emit a light pulse having a pulse width of less than or equal to 30 ns or even less than or equal to 20 ns.

As an irradiation pattern of the light source 110, for example, a pattern may be selected in which the distribution of intensity is uniform in the irradiation region. In that case, the subject 50 can be irradiated with light with spatially uniform illuminance, and the intensity of a detection signal at any one of the pixels of the image sensor 120 can be easily brought into a dynamic range.

### [1-2. Image Sensor 120]

The image sensor 120 receives light emitted from the light source 110 and reflected from the subject 50. The image sensor 120 has light detection cells arranged two-dimensionally and can acquire two-dimensional information regarding the subject 50 at a time. The image sensor 120 may be, for example, a freely chosen image pickup device such as a charge-coupled device (CCD) image sensor or a complementary metal oxide semiconductor (CMOS) image sensor. The image sensor 120 is an example of a sensor in the present disclosure. The sensor in the present disclosure is not limited to a sensor having light detection cells arranged two-dimensionally, and may be a sensor having light detection cells arranged one-dimensionally, for example. Moreover, in a use in which it is sufficient that only a piece of information be acquired, a light detector having a single photodetection device such as a photodetector may be used as the sensor. In that case, two photodetection devices next to each other are treated as the “first light detection cell” and the “second light detection cell”, and the technology in the present disclosure can be applied thereto.

The image sensor 120 according to the present embodiment has an electronic shutter. The electronic shutter is a circuit that controls exposure timings. The electronic shutter controls a one-time signal accumulation period, in which received light is converted into a valid electric signal and the valid electric signal is accumulated, and a period during which signal accumulation is stopped. The signal accumulation period is referred to as an “exposure period”. The time from when a one-time exposure period is completed to when the next exposure period is started is referred to as a “non-exposure period”. In the following, a state where exposure is performed may be expressed as “OPEN”, and a state where exposure is stopped may be expressed as “CLOSE”.

The image sensor 120 can adjust, using the electronic shutter, the exposure period and the non-exposure period with a subnanosecond accuracy such as an accuracy of 30 ps to 1 ns. Each exposure period may be set to, for example, a value greater than or equal to 1 ns and less than or equal to 30 ns.

In a case where the forehead of the subject 50 is irradiated with light to acquire information regarding, for example, the cerebral blood flow, the attenuation factor of light inside the living body is significantly large. For example, outgoing light may be attenuated to about one millionth the incident light. Thus, in order to detect the internal scattering component I2, there may be a case where the amount of light in irradiation with just one pulse is insufficient. In irradiation satisfying class 1 of the laser safety standards, in particular, the amount of light is very weak. In this case, the control circuit 132 causes the light source 110 to emit a light pulse multiple times and causes each light detection cell of the image sensor 120 to be exposed to light multiple times in synchronization with this emission. As a result, summation of signals is performed over multiple times, so that sensitivity can be improved.

In the following, an example of the configuration of the image sensor 120 will be described.

The image sensor 120 has pixels arranged two-dimensionally on the imaging plane. Each pixel has a photoelectric conversion element such as a photodiode and one or more charge accumulators.

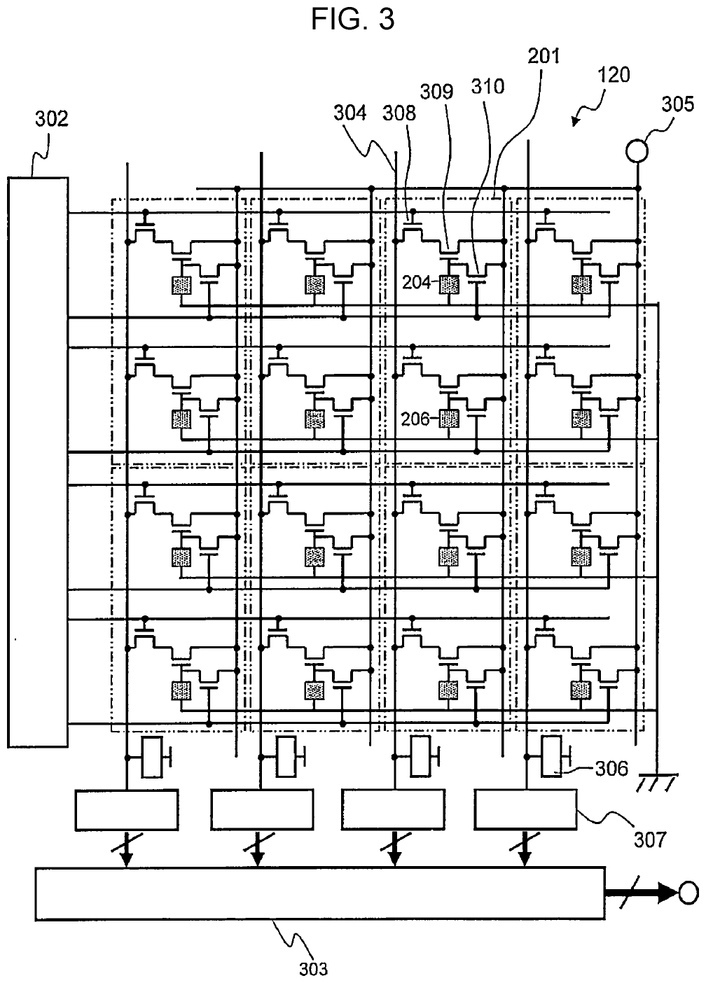

FIG. 3 is a diagram illustrating one example of the configuration of the image sensor 120. In FIG. 3, regions enclosed with dash-dot-dot lines each correspond to one pixel 201. Each pixel 201 includes a photoelectric conversion element that is not illustrated such as a photodiode. FIG. 3 illustrates only eight pixels arranged in two rows and four columns; however, many more pixels may actually be arranged. Each pixel 201 includes a first floating diffusion layer 204 and a second floating diffusion layer 206 serving as the charge accumulators. In this case, suppose that the light source 110 emits a first light pulse having a wavelength of greater than or equal to 650 nm and less than 805 nm and a second light pulse having a wavelength of greater than 805 nm and less than or equal to 950 nm. In the first floating diffusion layer 204, charge generated by receiving light of a reflected light pulse due to the first light pulse is accumulated. In the second floating diffusion layer 206, charge generated by receiving light of a reflected light pulse due to the second light pulse is accumulated. Signals accumulated in the first floating diffusion layer 204 and the second floating diffusion layer 206 are treated as if they were signals of two pixels of a general CMOS image sensor and are output from the image sensor 120. Note that, in a configuration with which detection of a reflected light pulse due to the first light pulse and detection of a reflected light pulse due to the second light pulse are switched on a frame basis and performed, each pixel 201 may include a single charge accumulator. Moreover, even in a configuration with which the light source 110 emits light having a single wavelength, each pixel 201 may include a single charge accumulator. In the present embodiment, one of two pixels 201 next to each other detects the surface reflection component I1 of the reflected light pulse, and the other one detects the internal scattering component I2 of the reflected light pulse.

In the example illustrated in FIG. 3, each pixel 201 has two signal detection circuits. Each signal detection circuit includes a source follower transistor 309, a row selection transistor 308, and a reset transistor 310. Each transistor is, for example, a field-effect transistor formed on a semiconductor substrate but is not limited to this transistor. As illustrated in the diagram, one out of an input terminal and an output terminal of the source follower transistor 309 is connected to one out of an input terminal and an output terminal of the row selection transistor 308. Typically, the above-described one out of the input terminal and the output terminal of the source follower transistor 309 is the source. Typically, the above-described one out of the input terminal and the output terminal of the row selection transistor 308 is the drain. The gate of the source follower transistor 309 serving as a control terminal is connected to the photodiode. Signal charge due to positive holes and electrons generated by the photodiode is accumulated in the floating diffusion layers serving as the charge accumulators between the photodiode and the source follower transistor 309.

Although not illustrated in FIG. 3, the first floating diffusion layer 204 and the second floating diffusion layer 206 are connected to the photodiode, which is not illustrated. A switch may be provided between the photodiode and each of the first floating diffusion layer 204 and the second floating diffusion layer 206. This switch switches, in accordance with a control signal from the control circuit 132, conduction states between the photodiode and each of the first floating diffusion layer 204 and the second floating diffusion layer 206. As a result, start and stoppage of accumulation of signal charge are controlled in each of the first floating diffusion layer 204 and the second floating diffusion layer 206. The electronic shutter according to the present embodiment has a mechanism for performing such exposure control.

The signal charge accumulated in each floating diffusion layer is read out by switching on the gate of the row selection transistor 308 using a row selection circuit 302. In this case, current flowing from a source follower power supply 305 into the source follower transistor 309 and a source follower load 306 is amplified in accordance with the signal potential of the floating diffusion layer. An analog signal based on this current read out from a vertical signal line 304 is converted by an analog-digital (AD) conversion circuit 307, which is connected on a column basis, into digital signal data. This digital signal data is read on a column basis by a column selection circuit 303 and is output from the image sensor 120. The row selection circuit 302 and the column selection circuit 303 read out one row and thereafter read out the next row and so forth, so that the row selection circuit 302 and the column selection circuit 303 read out information regarding signal charge in the floating diffusion layers of all the rows. After reading out all the signal charge, the control circuit 132 resets all the floating diffusion layers by switching on the gates of the reset transistors 310. As a result, image capturing of one frame is completed. By performing high-speed image capturing regarding frames repeatedly in the same way, image capturing of a series of frames using the image sensor 120 will be completed.

In the present embodiment, the image sensor 120, which is a CMOS type sensor, has been described as an example; however, the image sensor 120 may be an image sensor of another type. The image sensor 120 may be, for example, a CCD type image sensor, a single photon counting type device, or an amplification type image sensor such as an electron-multiplying CCD (EMCCD) or an intensified CCD (ICCD). Instead of the image sensor 120, in which the light detection cells are two-dimensionally arranged, a sensor may be used in which light detection cells are one-dimensionally arranged. Alternatively, sensors each having a single light detection cell may be used. In a case where a single pixel sensor is used, measurements can be performed only on one point on the living body; however, measurements can be performed at a high-speed rate.

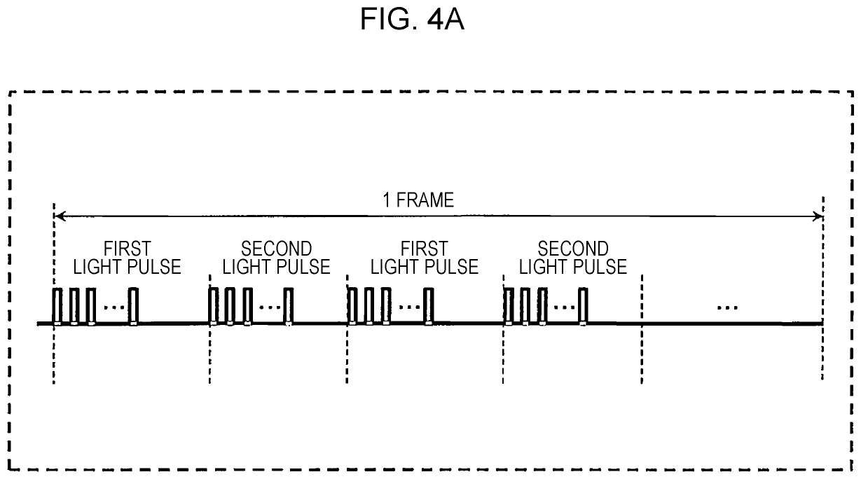

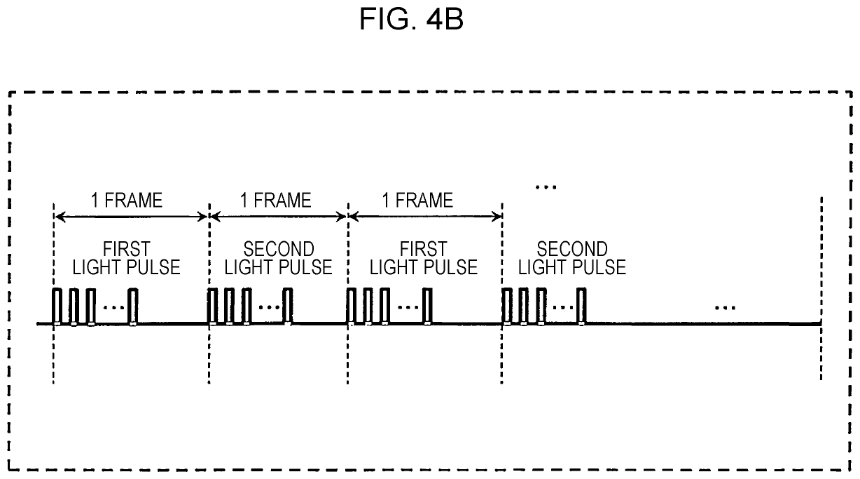

FIG. 4A is a diagram illustrating an example of an operation within one frame. As illustrated in FIG. 4A, within one frame, emission of first light pulses and emission of second light pulses may be switched multiple times in an alternating manner. By performing switching in this manner, a time difference between acquisition timings of detection images using two wavelengths can be reduced, and even in a case where the subject 50 is not stationary, image capturing using the first light pulses and image capturing using the second light pulses can be performed almost simultaneously.

FIG. 4B is a diagram illustrating another example of a detection operation using light having two wavelengths. As illustrated in FIG. 4B, detection of reflected light pulses due to first light pulses and detection of reflected light pulses due to second light pulses may be switched on a frame basis. Such an operation may be performed, for example, by switching emission of the first light pulses and emission of the second light pulses on a frame basis. In that case, each pixel 201 may have a single charge accumulator. With such a configuration, the number of charge accumulators of each pixel 201 can be reduced, and thus the size of each pixel 201 can be increased and sensitivity can be improved.

Note that the light source 110 may emit light having one wavelength. Even in that case, an approximate state of the brain activity can be estimated.

### [1-3. Electronic Circuit 130]

The electronic circuit 130 includes the control circuit 132, the signal processing circuit 134, and the memory 136. The control circuit 132 adjusts a time difference between an emission timing at which a light pulse is emitted from the light source 110 and a shutter timing of the image sensor 120. Herein, the time difference may also be referred to as a “phase difference”. The “emission timing” of the light source 110 refers to a timing at which a light pulse to be emitted from the light source 110 starts rising. The “shutter timing” refers to a timing at which exposure is started.

The control circuit 132 may be configured to remove an offset component from signals detected by the individual pixels of the image sensor 120. The offset component is a signal component caused by ambient light such as sunlight or light from a fluorescent light or disturbance light. By detecting a signal using the image sensor 120 in a state where driving of the light source 110 is switched off and light is not emitted from the light source 110, the offset component due to ambient light or disturbance light can be estimated.

The control circuit 132 may be, for example, a processor such as a central processing unit (CPU) or an integrated circuit such as a microcontroller in which a processor and a memory are built. The control circuit 132 adjusts the emission timings and the shutter timings by executing, for example, a computer program recorded in the memory 136 using the processor.

The signal processing circuit 134 is a circuit that processes an image signal output from the image sensor 120. The signal processing circuit 134 performs arithmetic processing such as image processing. The signal processing circuit 134 may be realized by, for example, a digital signal processor (DSP), a programmable logic device (PLD) such as a field-programmable gate array (FPGA), or a CPU or a processor for image processing (a graphics processing unit (GPU)). The signal processing circuit 134 executes processing to be described below by executing a computer program stored in the memory 136 using the processor.

The control circuit 132 and the signal processing circuit 134 may be one integrated circuit or may also be separated individual circuits. The signal processing circuit 134 may be, for example, a constituent element of an external apparatus such as a server provided at a remote place. In this case, through wireless communication or wired communication, the external apparatus such as a server interactively transmits data to and receives data from a measurement apparatus having the light source 110, the image sensor 120, and the control circuit 132. The signal processing circuit 134 generates, on the basis of a signal output from the image sensor 120, a surface layer signal reflecting the surface reflection component I1 and a deep-site signal reflecting the internal scattering component I2. Prior to this processing, the signal processing circuit 134 may estimate the offset component due to disturbance light and remove the offset component.

### [2. Example of Operation]

Next, an example of the operation of the present embodiment will be described.

In the present embodiment, the pixels of the image sensor 120 include first pixels P1 and second pixels P2. The control circuit 132 causes each first pixel P1 to detect a temporal front end portion of a reflected light pulse in a first exposure period. The control circuit 132 also causes each second pixel P2 to detect a temporal rear end portion of the reflected light pulse in a second exposure period. In this case, “detect a temporal front end portion” refers to detection of a component of at least part of the reflected light pulse in the rising period. In contrast, “detect a temporal rear end portion” refers to detection of a component of at least part of the reflected light pulse in the trailing period. As a result, the first pixels P1 acquire information regarding a relatively shallow site of the head, and the second pixels P2 acquire information regarding a relatively deep site of the head. With such a configuration, compared with a case where information regarding a shallow site and information regarding a deep site are acquired using one pixel, a biological signal can be generated with high temporal resolution.

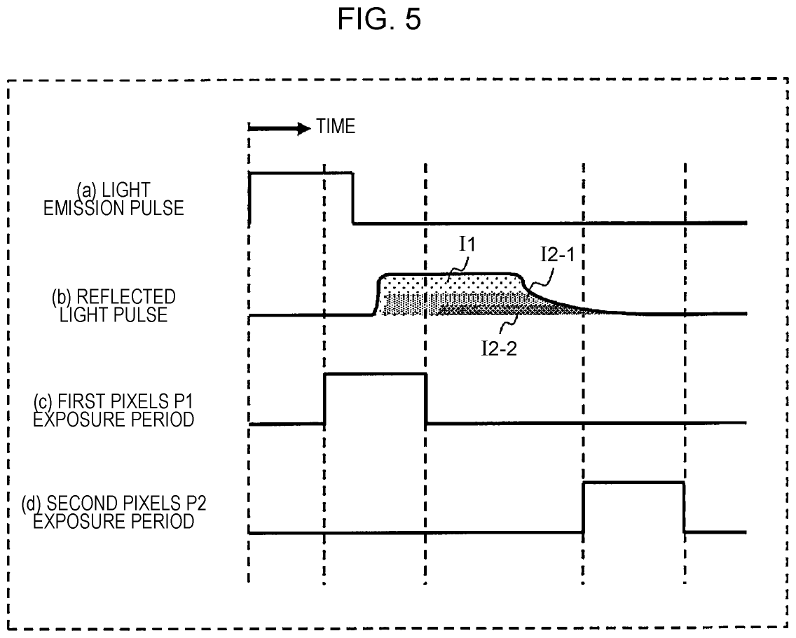

FIG. 5 is a schematic diagram for describing relationships between timings of a light emission pulse and a reflected light pulse and the first and second exposure periods. FIG. 5(a) illustrates an example of the waveform of a light emission pulse from the light source 110. FIG. 5(b) illustrates an example of the waveform of a reflected light pulse that reaches the image sensor 120. FIG. 5(c) illustrates an example of the first exposure period, during which the shutters of the first pixels P1 are open. FIG. 5(d) illustrates an example of the second exposure period, during which the shutters of the second pixels P2 are open.

The timing at which the reflected light pulse starts rising is later than the timing at which the light emission pulse of the light source 110 starts rising. The timing at which the reflected light pulse starts rising changes in accordance with the distance between the subject 50 and the image sensor 120. The intensity of the reflected light pulse gently falls in the trailing period due to the superimposition of impulse response waveforms on one another as described above. The later the shutter timing, the higher the proportion of the internal scattering component I2 included in the entirety of the acquired signal. The internal scattering component I2 mainly includes a scalp blood flow component I2-1 containing a high proportion of information regarding the scalp blood flow of a shallow forehead site and a cerebral blood flow component I2-2 containing a high proportion of information regarding the cerebral blood flow of a deep forehead site. In the present embodiment, as illustrated in FIG. 5(c) and FIG. 5(d), the pixels P1 are exposed to light with a phase containing a high proportion of the scalp blood flow component I2-1, and the pixels P2 are exposed to light with a phase containing a high proportion of the cerebral blood flow component I2-2.

In the present embodiment, after the first exposure period ends, the second exposure period is started. The first exposure period is set to include at least part of the rising period of a reflected light pulse reaching the image sensor 120 in a case where a target is positioned at a preset distance from the measurement apparatus 100. The first exposure period may be set to include the entirety of the rising period of the reflected light pulse or may be set to include only part of the rising period. The first exposure period may be set to start, for example, before the rising period of the reflected light pulse ends and end before the trailing period starts. The second exposure period is set to include at least part of the trailing period of a reflected light pulse reaching the image sensor 120 in a case where a target is positioned at a preset distance from the measurement apparatus 100. The second exposure period may be set to start, for example, in the period from the start of the trailing period of the reflected light pulse and to the end of the trailing period. The first exposure period and the second exposure period may partially overlap one another.

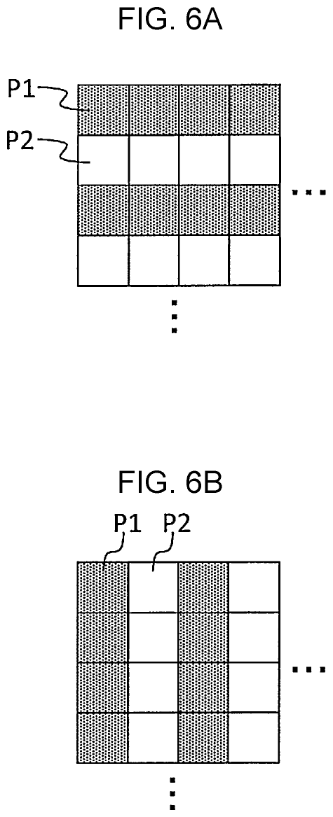

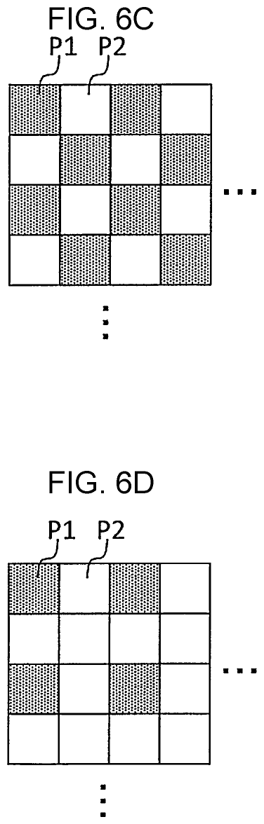

FIGS. 6A to 6D are diagrams illustrating examples of an arrangement pattern of the first pixels P1 and the second pixels P2 of the image sensor 120, the first pixels P1 and the second pixels P2 being two kinds of pixels having different exposure periods. By changing the arrangement pattern, optimal information can be acquired depending on the purpose of the measurement.

FIG. 6A illustrates an example of an arrangement pattern in which rows formed by the first pixels P1 and rows formed by the second pixels P2 are aligned in an alternating manner. In this case, the exposure periods for the even rows have different phases from those for the odd rows. This arrangement is useful in a case where it is desired to capture a steep change in the horizontal direction. For example, as in the case where it is desired to detect a phenomenon in which the left brain is activated but the right brain does not change, the arrangement of FIG. 6A is effective in a case where it is desired to compare differences between signals of the left and right portions of the forehead.

FIG. 6B illustrates an example of an arrangement pattern in which columns formed by the first pixels P1 and columns formed by the second pixels P2 are aligned in an alternating manner. In this case, the exposure periods for the even columns have different phases from those for the odd columns. This arrangement is useful in a case where it is desired to capture a steep change in the vertical direction. For example, as in the case where it is desired to detect a phenomenon in which changes in the cerebral blood flow and changes in the blood flow in the skin of part of the face have different tendencies, the arrangement of FIG. 6B is effective in a case where it is desired to compare differences between signals of the upper and lower portions of the forehead.

FIG. 6C illustrates an example in which the first pixels P1 and the second pixels P2 are arranged in a checkerboard pattern. With this arrangement pattern, signals of the entire forehead can be evenly evaluated in any of the up-and-down direction and the left-and-right direction.

The arrangement pattern of the pixels P1 and P2 may be a pattern in which a pixel block having N rows×M columns (N and M are freely chosen natural numbers) serving as a unit is arranged repeatedly. For example, as illustrated in FIG. 6D, a pattern may be used in which 2-row-by-2-column pixels serving as a unit are arranged repeatedly. In the example of FIG. 6D, the number of second pixels P2 is three times as many as that of first pixels P1. As described above, the signal of the internal scattering component I2 is very weak. Thus, an SN ratio can be improved by making the number of second pixels P2 larger than the number of first pixels P1 and calculating the average of the signal values of the second pixels P2.

Moreover, the first pixels P1 and the second pixels P2 may be switched in the example illustrated in FIG. 6D. In that case, the number of first pixels P1 is larger than the number of second pixels P2. The first pixels P1 contain a high proportion of surface layer information regarding the subject 50 such as the facial appearance, and thus the accuracy of processing can be improved, the processing being for example analysis of facial expressions or lines of sight of the subject 50 or estimation of the magnitude or speed of the body movement.

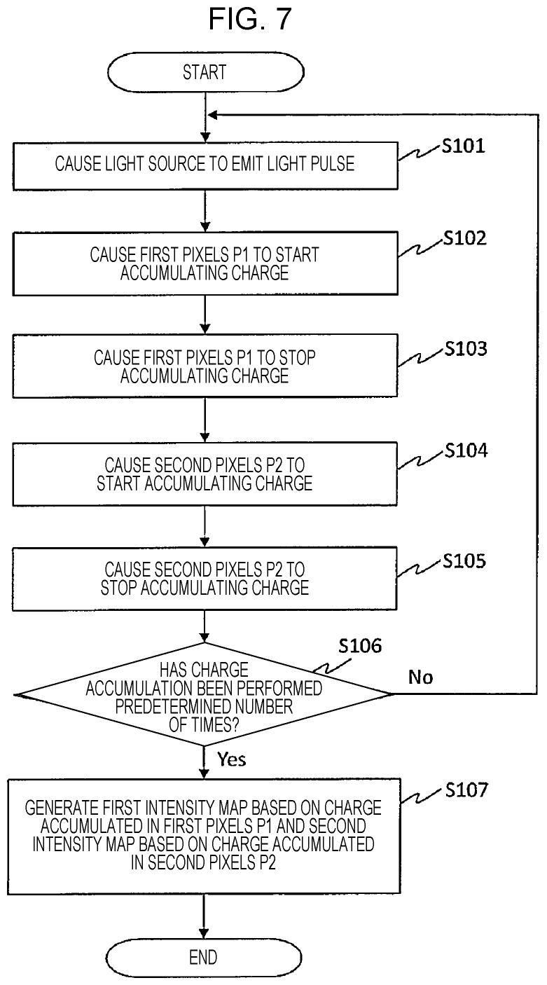

FIG. 7 is a flow chart illustrating the summary of an operation performed by the electronic circuit 130 according to the present embodiment. In this case, for brevity, suppose a configuration with which the light source 110 emits light having one wavelength. First, the control circuit 132 causes the light source 110 to emit a light pulse only during a predetermined time period (step S101). The control circuit 132 causes the image sensor 120 to start accumulating charge in each first pixel P1 at a first timing (step S102). Next, the control circuit 132 causes the image sensor 120 to stop accumulating charge in each first pixel P1 at a second timing (step S103). The first exposure period is the period from the first timing to the second timing. Through the operations in steps S102 and S103, charge corresponding to the intensity of the front end portion of a reflected light pulse is accumulated in each first pixel P1. Subsequently, the control circuit 132 causes the image sensor 120 to start accumulating charge in each second pixel P2 at a third timing (step S104). Next, the control circuit 132 causes the image sensor 120 to stop accumulating charge in each second pixel P2 at a fourth timing (step S105). The second exposure period is the period from the third timing to the fourth timing. Through the operations in steps S104 and S105, charge corresponding to the intensity of the rear end portion of the reflected light pulse is accumulated in each second pixel P2.

Subsequently, the control circuit 132 determines whether the number of times the above-described charge accumulation has been executed reaches a predetermined number of times (step S106). In a case where No is obtained in this determination, the operation from steps S101 to S105 is repeated until Yes is obtained in this determination. When Yes is obtained in step S106, the signal processing circuit 134 reads out signals of charge accumulated in the individual pixels of the image sensor 120. The signal processing circuit 134 generates and outputs a first intensity map based on the charge accumulated in the first pixels P1 and a second intensity map based on the charge accumulated in the second pixels P2 (step S107).

In step S107, the signal processing circuit 134 generates image data of the respective intensity maps by extracting data of pixels of the same kind from image data acquired by the image sensor 120 on the basis of an arrangement pattern, examples of which are illustrated in FIGS. 6A to 6D, and collecting the data of the pixels of the same kind. Specifically, the signal processing circuit 134 extracts only data of the first pixels P1 from the acquired image data to generate the first intensity map and extracts only data of the second pixels P2 to generate the second intensity map. For example, in the example of FIG. 6A, the first intensity map is generated from the data of the first pixels P1 of the odd rows, and the second intensity map is generated from the data of the second pixels P2 of the even rows. In this case, pixels that do not have data may be interpolated on the basis of the data of their surrounding pixels. The first intensity map is an example of the surface layer data and may be, for example, image data representing the facial appearance of the subject 50 or the distribution of the scalp blood flow. The second intensity map is an example of the deep-site data and may be, for example, image data representing the distribution of the cerebral blood flow of the subject 50. The signal processing circuit 134 may remove a scalp blood flow component, which causes an artifact, by performing a computation to separate characteristics of the intensity map of the surface layer from characteristics of the intensity map of the deep site. Through this processing, information regarding changes in the cerebral blood flow of the subject 50 over time can be acquired with high accuracy and high temporal resolution.

In the following, an example of the computation for separating the characteristics of the intensity map of the surface layer from the characteristics of the intensity map of the deep site will be described. In this case, as an example, the case will be described where the charge accumulation operation from steps S101 to S106 is performed on the first light pulse having a wavelength greater than or equal to 650 nm and less than 805 nm and on the second light pulse having a wavelength greater than 805 nm and less than or equal to 950 nm. In that case, each pixel in each of the first intensity map and the second intensity map has a value of the intensity of reflected light due to the first light pulse (hereinafter referred to as a “first value”) and a value of the intensity of reflected light due to the second light pulse (hereinafter referred to as a “second value”). The signal processing circuit 134 calculates, from each intensity map, the amounts of change in the concentration of oxygenated hemoglobin (HbO₂) and that of deoxygenated hemoglobin (Hb) from the initial values. Specifically, the signal processing circuit 134 calculates Δ[HbO₂]_SurfaceLayer, an amount of change from the initial value of the concentration of HbO₂ in the surface layer, and Δ[Hb]_SurfaceLayer, an amount of change from the initial value of the concentration of Hb in the surface layer, by solving preset simultaneous equations using the first values and the second values of the individual pixels in the first intensity map. Similarly, the signal processing circuit 134 calculates Δ[HbO₂]_DeepSite, an amount of change from the initial value of the concentration of HbO₂ in the deep site, and Δ[Hb]_DeepSite, an amount of change from the initial value of the concentration of Hb in the deep site, by solving preset simultaneous equations using the first values and the second values of the individual pixels in the second intensity map. The signal processing circuit 134 calculates, using the following equations, Δ[HbO₂]_CerebralBloodFlow and Δ[Hb]_CerebralBloodFlow, amounts of change from the initial values of the respective concentrations of HbO₂ and Hb in the cerebral blood flow.

Δ[HbO₂]_CerebralBloodFlow=Δ[HbO₂]_DeepSite−kΔ[HbO₂]_SurfaceLayer

Δ[Hb]_CerebralBloodFlow=Δ[Hb]_DeepSite−kΔ[Hb]_SurfaceLayer

In this case, a coefficient k is a known value calculated in advance using a model that imitates a person (a phantom). The coefficient k is the ratio of the “volume of the scalp blood flow component of the deep site” to the “volume of the scalp blood flow of the surface layer”. That is, k=(the volume of the scalp blood flow component of the deep site)/(the volume of the scalp blood flow of the surface layer).

By performing the operation illustrated in FIG. 7, two kinds of image data, which are image data of the surface layer and that of the deep site, can be generated on a frame basis. The electronic circuit 130 may generate data of a moving image by repeating this series of operations multiple times. Note that light emission and charge accumulation do not have to be performed multiple times and are performed as needed.

The signal processing circuit 134 according to the present embodiment can generate, on the basis of image data output on a frame basis from the image sensor 120, moving image data representing changes in the cerebral blood flow over time and moving image data representing changes in the facial appearance over time. The signal processing circuit 134 may generate not only such moving image data but also other information. For example, living body information such as the blood flow volume, blood pressure, and blood oxygen saturation in the brain or the heart rate may be generated in synchronization with other apparatuses. Moreover, living body information such as the skin blood flow volume, the heart rate, or the amount of sweat may also be generated on the basis of the surface reflection component I1 detected by the individual pixels included in the image sensor 120.

It is known that there is a close relationship between changes in cerebral blood flow volume or in a blood component such as hemoglobin and the nerve activity of a person. For example, the activity of nerve cells of a person changes in accordance with the interest level of the person, so that the cerebral blood flow volume or a component in the blood changes. Thus, in a case where living body information such as the cerebral blood flow volume or facial appearance information can be measured, the psychological state or body condition of the user can be estimated. The psychological state of the user may be, for example, feelings, emotions, health conditions, or a thermal sense. The feelings may include, for example, a feeling such as comfort or discomfort. The emotions may include, for example, an emotion such as peace of mind, anxiety, sadness, or anger. The health conditions may include, for example, the state of healthiness or the state of weariness. The thermal sense may include, for example, a sense such as hot, cold, or muggy. As derivatives from these, measures representing the level of brain activity such as the level of interest, the level of skill, the level of proficiency, and the level of concentration may also be included in psychological states. Furthermore, a body condition such as the level of fatigue, the level of sleepiness, or the level of intoxication under the influence of alcohol is also included in targets to be estimated by the signal processing circuit 134. The signal processing circuit 134 can estimate the user's psychological state or body condition on the basis of at least one of a change in the cerebral blood flow state, a change in the scalp blood flow state, or a change in the facial appearance and output a signal representing the estimation result.

### Second Embodiment

Next, a second embodiment, which is an example, of the present disclosure will be described. In the present embodiment, the pixels of the image sensor 120 include three kinds of pixels, which are the first pixels P1, the second pixels P2, and third pixels P3. Different exposure periods are set for these first pixels P1, second pixels P2, and third pixels P3. According to the present embodiment, information regarding changes over time in the appearance, scalp blood flow, and cerebral blood flow of the subject 50 can be acquired with high temporal resolution.

In the following, points that are different from those of the first embodiment will be mainly described, and redundant description will be omitted.

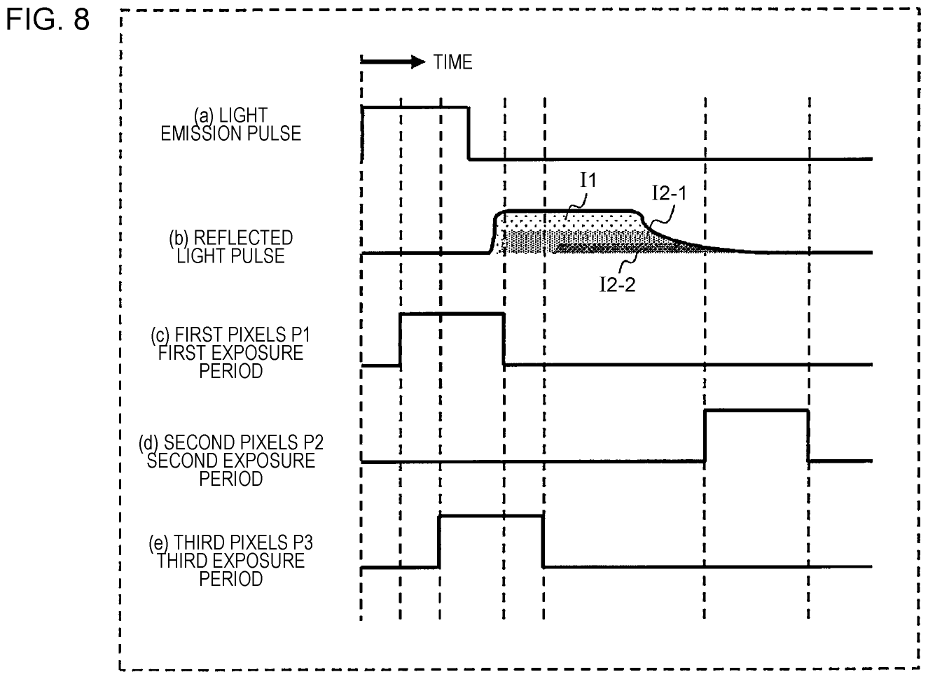

FIG. 8 is a schematic diagram for describing relationships between timings of a light emission pulse and a reflected light pulse according to the present embodiment and first to third exposure periods. FIG. 8(a) illustrates an example of the waveform of a light emission pulse from the light source 110. FIG. 8(b) illustrates an example of the waveform of a reflected light pulse that reaches the image sensor 120. FIG. 8(c) illustrates an example of the first exposure period, during which the shutters of the first pixels P1 are open. FIG. 8(d) illustrates an example of the second exposure period, during which the shutters of the second pixels P2 are open. FIG. 8(e) illustrates an example of the third exposure period, during which the shutters of the third pixels P3 are open.

The first pixels P1 are exposed to light with a phase containing a high proportion of the surface reflection component I1. The second pixels P2 are exposed to light with a phase containing a high proportion of the cerebral blood flow component I2-2. The third pixels P3 are exposed to light with a phase containing a high proportion of the scalp blood flow component I2-1.

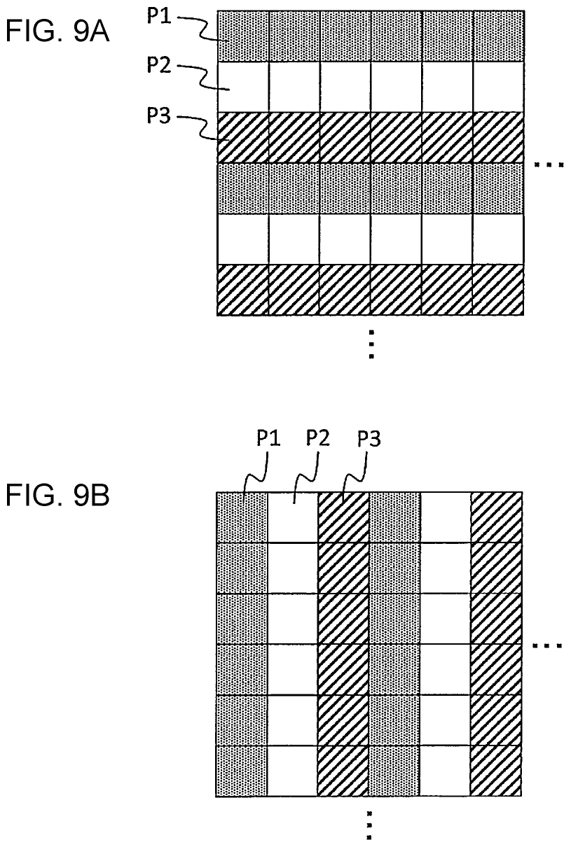

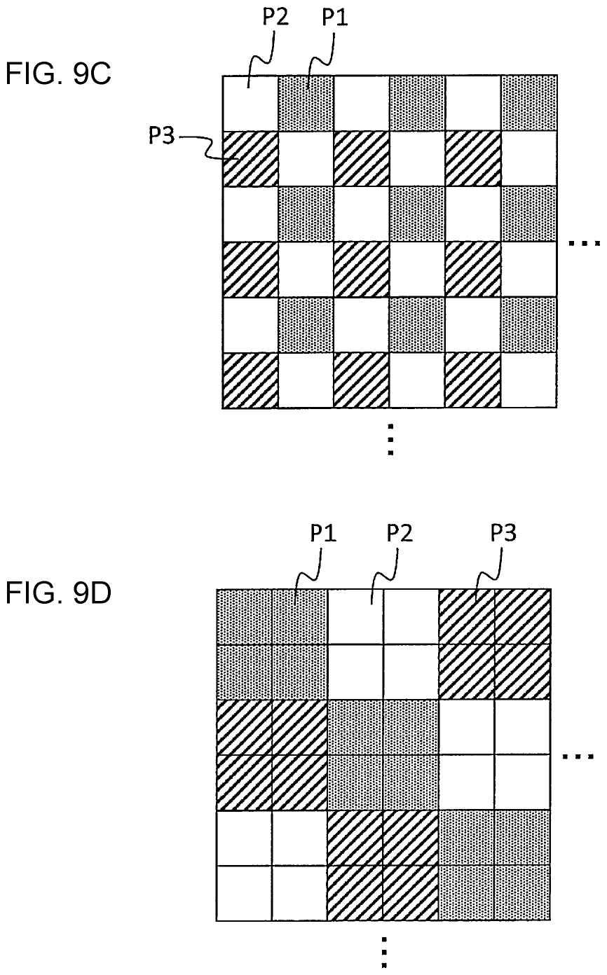

FIGS. 9A to 9D are diagrams illustrating examples of an arrangement pattern of the first pixels P1, the second pixels P2, and the third pixels P3 of the image sensor 120, the first pixels P1, the second pixels P2, and the third pixels P3 being three kinds of pixels having different exposure periods. Even in the present embodiment, by changing the arrangement pattern, optimal information can be acquired depending on the purpose of the measurement.

FIG. 9A illustrates an example of an arrangement pattern in which rows formed by the first pixels P1, rows formed by the second pixels P2, and rows formed by the third pixels P3 are aligned in an alternating manner. This arrangement is useful in a case where it is desired to capture a steep change in the horizontal direction. For example, as in the case where it is desired to detect a phenomenon in which the left brain is activated but the right brain does not change, the arrangement of FIG. 9A is effective in a case where it is desired to compare differences between signals of the left and right portions of the forehead.

FIG. 9B illustrates an example of an arrangement pattern in which columns formed by the first pixels P1, columns formed by the second pixels P2, and columns formed by the third pixels P3 are aligned in an alternating manner. This arrangement is useful in a case where it is desired to capture a steep change in the vertical direction. For example, as in the case where it is desired to detect a phenomenon in which changes in the cerebral blood flow and changes in the blood flow in the skin of part of the face have different tendencies, the arrangement of FIG. 9B is effective in a case where it is desired to compare differences between signals of the upper and lower portions of the forehead.

FIG. 9C illustrates an example in which the first pixels P1, the second pixels P2, and the third pixels P3 are arranged in a checkerboard pattern. With this arrangement pattern, signals of the entire forehead can be evenly evaluated in any of the up-and-down direction and the left-and-right direction.

As illustrated in FIG. 9D, the arrangement pattern of the first pixels P1, the second pixels P2, and the third pixels P3 may be a pattern in which the first pixels P1, the second pixels P2, and the third pixels P3 are arranged in an alternating manner in units of a pixel block having N rows×M columns (N and M are freely chosen natural numbers).

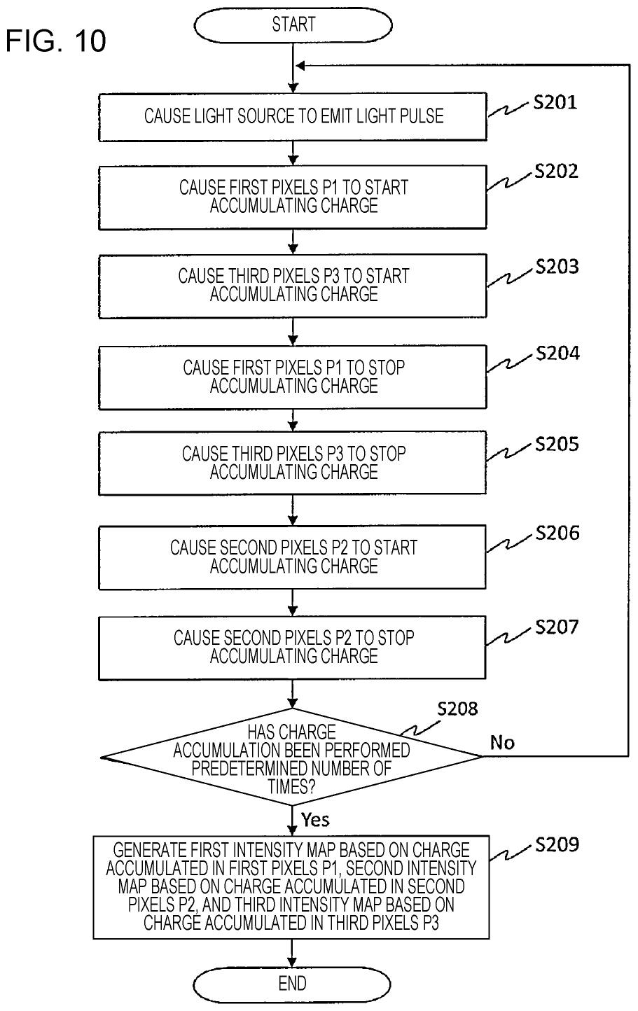

FIG. 10 is a flow chart illustrating the summary of an operation performed by the electronic circuit 130 according to the second embodiment. For brevity, an example in which light to be used has one wavelength will be described also in this case. First, the control circuit 132 causes the light source 110 to emit a light pulse only during a predetermined time period (step S201). The control circuit 132 causes the image sensor 120 to start accumulating charge in each first pixel P1 (step S202). Next, the control circuit 132 causes the image sensor 120 to start accumulating charge in each third pixel P3 (step S203). When a time corresponding to the first exposure period elapses from step S202, the control circuit 132 causes the image sensor 120 to stop accumulating charge in each first pixel P1 (step S204). When a time corresponding to the third exposure period elapses from step S203, the control circuit 132 causes the image sensor 120 to stop accumulating charge in each third pixel P3 (step S205). After a predetermined time has elapsed from step S204, the control circuit 132 causes the image sensor 120 to start accumulating charge in each second pixel P2 (step S206). When a time corresponding to the second exposure period elapses from step S206, the control circuit 132 causes the image sensor 120 to stop accumulating charge in each second pixel P2 (step S207).

Subsequently, the control circuit 132 determines whether the number of times the above-described charge accumulation has been executed reaches a predetermined number of times (step S208). In a case where No is obtained in this determination, the operation from steps S201 to S207 is repeated until Yes is obtained in this determination. When Yes is obtained in step S208, the signal processing circuit 134 reads out signals of charge accumulated in the individual pixels of the image sensor 120. The signal processing circuit 134 generates and outputs a first intensity map based on the charge accumulated in the first pixels P1, a second intensity map based on the charge accumulated in the second pixels P2, and a third intensity map based on the charge accumulated in the third pixels P3 (step S209).

By performing the operation illustrated in FIG. 10, three kinds of image data can be generated on a frame basis, three kinds of image data being data reflecting the intensity distribution of the surface reflection component I1, data reflecting the intensity distribution of the scalp blood flow component I2-1, and data reflecting the intensity distribution of the cerebral blood flow component I2-2. The electronic circuit 130 may generate data of a moving image by repeating this series of operations multiple times. Note that, even in the present embodiment, light emission and charge accumulation do not have to be performed multiple times and are performed as needed.

As described above, according to the present embodiment, appearance information, scalp blood flow information, and cerebral blood flow information can be acquired with high temporal resolution.

In the examples of FIGS. 8 and 10, the third exposure period is started before the first exposure period ends. However, the third exposure period is not necessarily started before the first exposure period ends. For example, the third exposure period may be started after the first exposure period ends. Moreover, the third exposure period and the second exposure period may partially overlap one another.

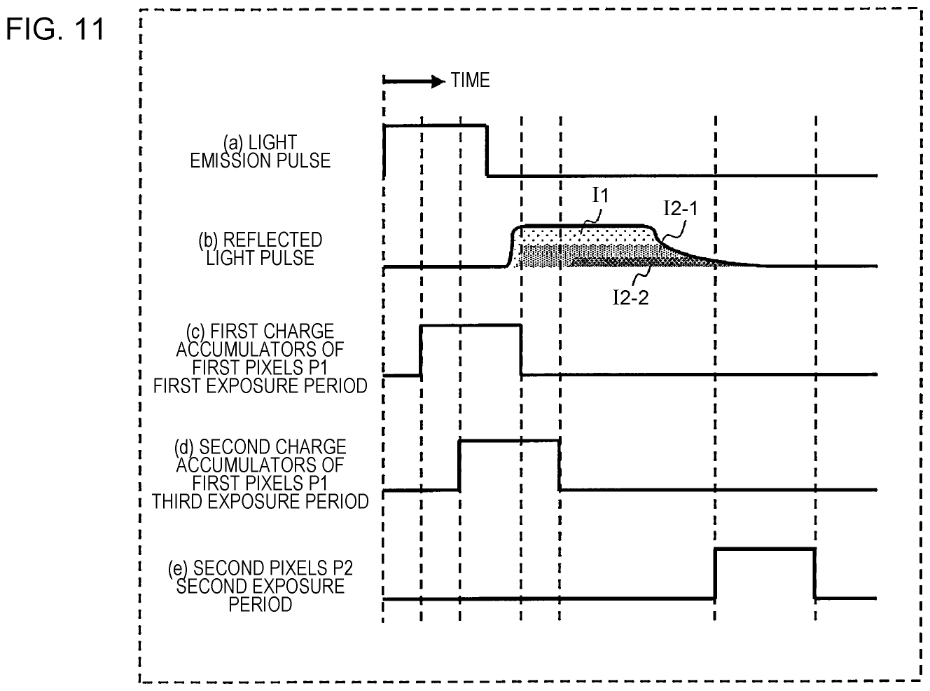

FIG. 11 is a timing chart illustrating a modification of the present embodiment. In this modification, the third pixels P3 are not provided, and the first pixels P1 are exposed to light in a first exposure period and a third exposure period. Thus, each first pixel P1 has charge accumulators as in the example illustrated in FIG. 3. The first exposure period and the third exposure period are allocated to a first charge accumulator and a second charge accumulator of each first pixel P1, respectively. As a result, three kinds of biological signals can be acquired using two kinds of pixels. In this case, the arrangement of the first pixels P1 and the second pixels P2 may be, for example, any one of the arrangements illustrated in FIGS. 6A to 6D. Measurements can be performed with higher spatial resolution than in a case where three kinds of pixels are set.

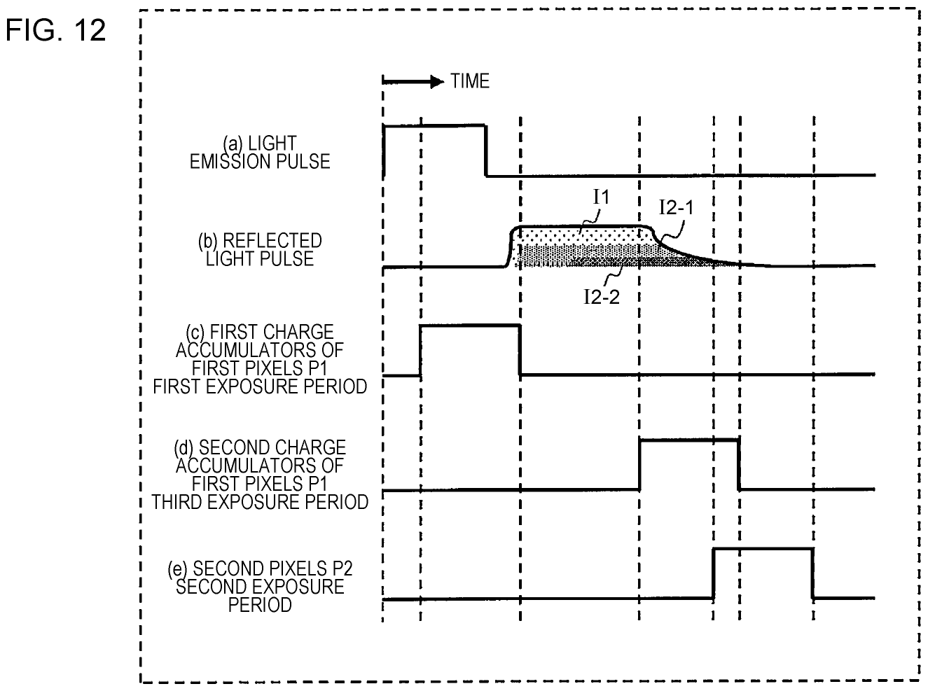

FIG. 12 is a timing chart illustrating another modification of the present embodiment. In this example, unlike the example illustrated in FIG. 11, a third exposure period is started immediately before the start of the trailing period of a reflected light pulse. A first exposure period is started, for example, before the start of the rising period of the reflected light pulse and ends immediately after the end of the rising period as in the example illustrated in FIG. 11. The signal processing circuit 134 in this example generates data representing the distance from the image sensor 120 to the subject 50 on the basis of first signals based on charge accumulated in the first charge accumulators of the first pixels P1 in the first exposure period and third signals based on charge accumulated in the second charge accumulators of the first pixels P1 in the third exposure period, and outputs the data.

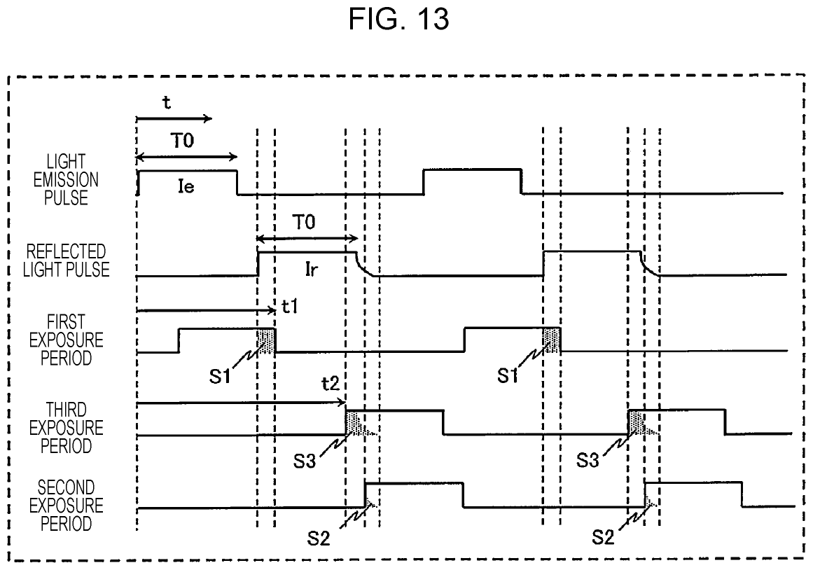

FIG. 13 is a diagram for describing the principle of a distance calculation. FIG. 13 illustrates, as an example, a light emission pulse Ie, a reflected light pulse Ir, a first exposure period, a second exposure period, and a third exposure period. A duration of time of the light emission pulse and that of the reflected light pulse are denoted by T0. The time from when the light source 110 starts emitting a light pulse to when the first exposure period ends is denoted by t1, and the time from when the light source 110 starts emitting a light pulse to when the third exposure period is started is denoted by t2. In this example, 0<t2−t1<T0 holds. In the first exposure period, charge generated by exposure to the front end portion of the reflected light pulse is accumulated. In contrast, in the third exposure period, charge generated by exposure to the rear end portion of the reflected light pulse is accumulated. A signal representing the amount of charge accumulated in the first exposure period is denoted by S1, and a signal representing the amount of charge accumulated in the third exposure period is denoted by S3. The intensity of each of the signals S1 and S3 changes in accordance with a distance z between the subject 50 and the image sensor 120. The distance z can be calculated using Equation (1) below.

$$z = c \frac{\{ {T}_{0} - ( {t}_{2} - {t}_{1} ) \}}{2} \times \frac{{S}_{3}}{{S}_{1} + {S}_{3}} \qquad ( 1 )$$

In this case, c (≈3.0×10⁸ m/s) represents the velocity of light.

The signal processing circuit 134 can calculate the distance z from the signals S1 and S3 on the basis of Equation (1).

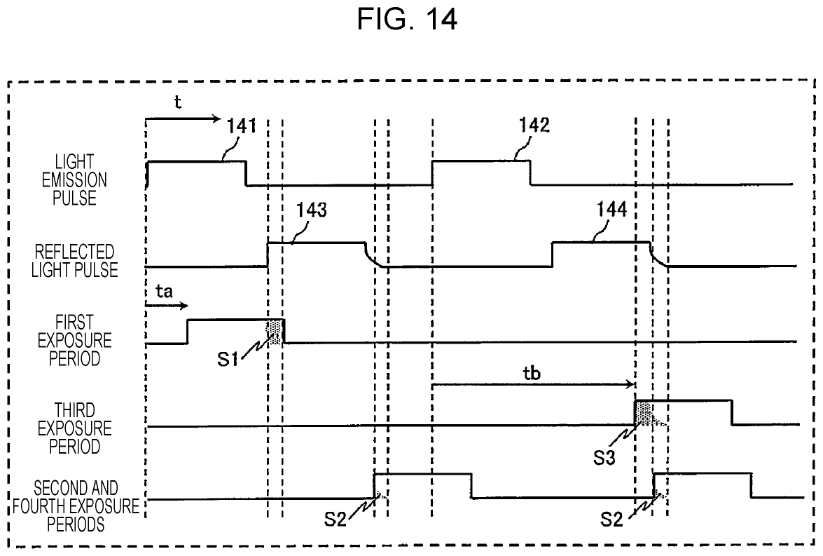

FIG. 14 is a diagram illustrating yet another modification. In this example, the control circuit 132 causes the light source 110 to emit a second light pulse 142 after the control circuit 132 causes the light source 110 to emit a first light pulse 141. Regarding first reflected light 143 generated due to the first light pulse 141, charge is accumulated in the first charge accumulator of each first pixel P1 in a first exposure period, and charge is accumulated in the charge accumulator of each second pixel P2 in a second exposure period. As a result, the signal S1 is output from the first charge accumulator of each first pixel P1, and the signal S2 is output from the charge accumulator of each second pixel P2. Regarding second reflected light 144 generated due to the second light pulse 142, charge is accumulated in the second charge accumulator of each first pixel P1 in a third exposure period, and charge is accumulated in the charge accumulator of each second pixel P2 in a fourth exposure period. As a result, the signal S3 is output from the second charge accumulator of each first pixel P1, and the signal S2 is output from the charge accumulator of each second pixel P2. In this case, a length to from the time at which emission of the first light pulse 141 is started to the time at which the first exposure period is started is shorter than a length tb from the time at which emission of the second light pulse 142 is started to the time at which the third exposure period is started. The signal processing circuit 134 generates, from the signals S1 and S3 and on the basis of Equation (1) described above, data representing the distance z from the image sensor 120 to the target and outputs the data.

By using the method illustrated in FIG. 13 or 14, the distance from the image sensor 120 to a target can be calculated on a pixel basis. The electronic circuit 130 can generate data of a moving image representing changes over time in each of the two-dimensional distribution of distance and the two-dimensional distribution of cerebral blood flows by continuously repeating an operation for acquiring distance information and cerebral blood information from each pixel. The electronic circuit 130 can adjust, on the basis of the distance information acquired from each pixel by performing each measurement, timings of the first to fourth exposure periods on a pixel basis in the next measurement. By performing such an adjustment, a cerebral blood flow signal can be acquired with higher accuracy.

The electronic circuit 130 may correct the cerebral blood information using the distance information acquired by using the above-described method. Light emitted from the light source 110 has a unique illuminance distribution corresponding to characteristics of the light source 110. The level of an acquired cerebral blood flow signal changes in accordance with the spatial illuminance distribution of light emitted by the light source 110 and varies depending on the position of a measurement point. In a case where the subject 50 has moved while the measurement apparatus 100 is repeatedly acquiring a cerebral blood flow signal of the subject 50, the distance from the measurement apparatus 100 to the measurement point changes, and thus the level of the acquired cerebral blood flow signal changes. In order to obtain a preferable measurement result, it is important to suppress the effect of this change. Thus, the electronic circuit 130 performs a cerebral blood flow measurement and a distance measurement at the same time and can correct the cerebral blood flow signal at each measurement point on the basis of the measured distance from the measurement apparatus 100 to the measurement point.

In the following, with reference to FIGS. 15 to 17, an operation for correcting a cerebral blood flow signal on the basis of the distance will be described. In this specification, the “cerebral blood flow signal” refers to a signal representing the state of the subject's cerebral blood flow and is a signal representing the intensity of an internal scattering component of light from a to-be-measured site or a signal generated by processing the signal.

FIG. 15 is a diagram illustrating examples of changes over time in three kinds of images generated by the electronic circuit 130. In this example, the electronic circuit 130 repeats an operation for generating three kinds of images illustrated in FIG. 15, which are a first image (a), a third image (b), and a second image (c), by executing the operation illustrated in FIG. 13 or 14. FIG. 15 illustrates an example of three kinds of images in an initial frame and an example of three kinds of images in the current frame. Movement of the subject 50 (that is, body movement) and changes in the blood flow of their scalp and brain occur over time, and changes may occur in the luminance distribution of each image due to the effects of these movement and changes.

In the example illustrated in FIG. 15, the control circuit 132 of the electronic circuit 130 causes the two kinds of pixels (that is, the first pixels P1 and the second pixels P2) of the image sensor 120 to be exposed to light in the three kinds of exposure periods illustrated in FIG. 13 or 14. The arrangement of the pixels may be, for example, any one out of the arrangements illustrated in FIGS. 6A to 6D. The signal processing circuit 134 of the electronic circuit 130 extracts, from the signal acquired from the image sensor 120, a signal of the first charge accumulator of each first pixel P1, a signal of the second charge accumulator of each first pixel P1, and a signal of the charge accumulator of each second pixel P2, and generates the first image (a), the third image (b), and the second image (c) on the basis of these signals. The first charge accumulator of each first pixel P1 detects the front end portion of a reflected light pulse in the first exposure period and generates a first signal. The signal processing circuit 134 generates the first image (a) on the basis of the first signals. The second charge accumulator of each first pixel P1 detects a component corresponding to the period from immediately before the start of the trailing period of the reflected light pulse to the end of the trailing period in the third exposure period and generates a third signal. The signal processing circuit 134 generates the third image (b) on the basis of the third signals. The charge accumulator of each second pixel P2 detects the rear end portion of the reflected light pulse in the second exposure period and generates a second signal. The signal processing circuit 134 generates the second image (c) on the basis of the second signals. The second image (c) represents the two-dimensional distribution of an internal scattering component scattered in the cerebral blood flow of the subject 50.

The signal processing circuit 134 measures a distance on a pixel basis by performing the calculation expressed in Equation (1) described above using the first signal and the third signal at each pixel. The signal processing circuit 134 generates a distance image on the basis of the calculated distance of each pixel. Furthermore, the signal processing circuit 134 corrects the second image (c) on the basis of the distance image.

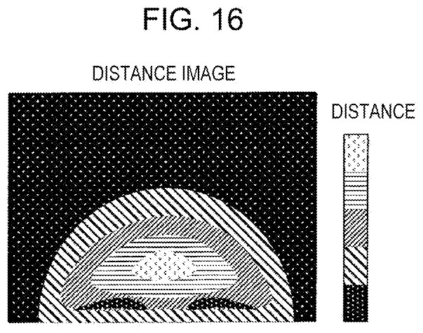

FIG. 16 is a diagram schematically illustrating an example of the distance image generated on the basis of the first image (a) and the third image (b). The signal processing circuit 134 corrects the second image (c) on the basis of such a distance image. More specifically, on the basis of the distance image and data prepared in advance and representing the illuminance distribution of the light source 110, the signal processing circuit 134 generates a correction value image for correcting the second image (c). Using the correction value image, the signal processing circuit 134 performs a correction to remove the effect of the body movement from the second image (c).

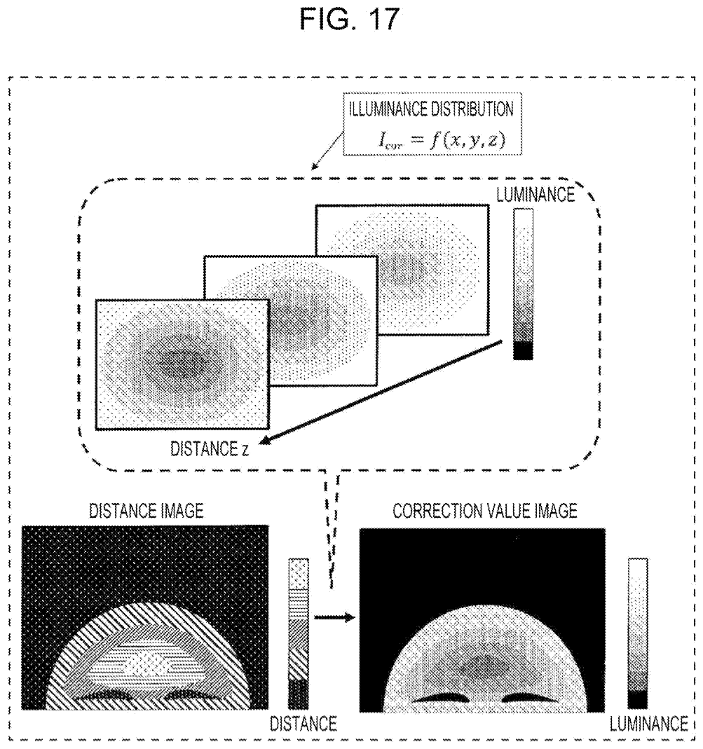

FIG. 17 is a diagram schematically illustrating the procedure of an operation for generating a correction value image to be used in correction processing performed by the signal processing circuit 134. To perform a correction, information representing the illuminance distribution of the light source 110 that has been acquired in advance through calibration is used. The calibration is performed before the subject 50 is measured. The information representing the illuminance distribution represents the distribution of spatial illuminance that light emitted by the light source 110 has and is expressed as Equation (2) below.

I_{cor}=f(x,y,z) (2)

In this case, I_{cor} represents luminance at a position (x, y, z) in the space, (x, y) represents the position of a pixel in an image, and z represents the distance calculated using Equation (1). In the calibration, for example, an object such as a white board is treated as a measurement target and exposure is performed at the timings illustrated in FIG. 12. It is desirable that a flat plate phantom having an absorption coefficient and a scattering coefficient whose values are close to those of a person be used as the measurement target. While changing the distance between the measurement apparatus 100 and the white board, distance images and second images (c) are acquired at distances. At each distance, information regarding the distance z and the luminance I_{cor} at a pixel position (x, y) of the distance image and the second image (c) is acquired. The function expressed in Equation (2) is obtained using the acquired data of the distance z and the luminance I_{cor} and by performing interpolation and extrapolation between data points. Interpolation between the data points is not limited to interpolation and extrapolation, and various regression methods may be applied to the interpolation between the data points. This illuminance distribution may be acquired every time the subject is measured or may be acquired only once at the beginning. The function expressed in Equation (2) may be recorded, for example, in a table format on a storage medium such as the memory 136.

The signal processing circuit 134 can generate a correction value image as illustrated in the bottom right of FIG. 17 by entering information regarding the distance image acquired from the subject 50 by using the above-described method into the function expressed in Equation (2).

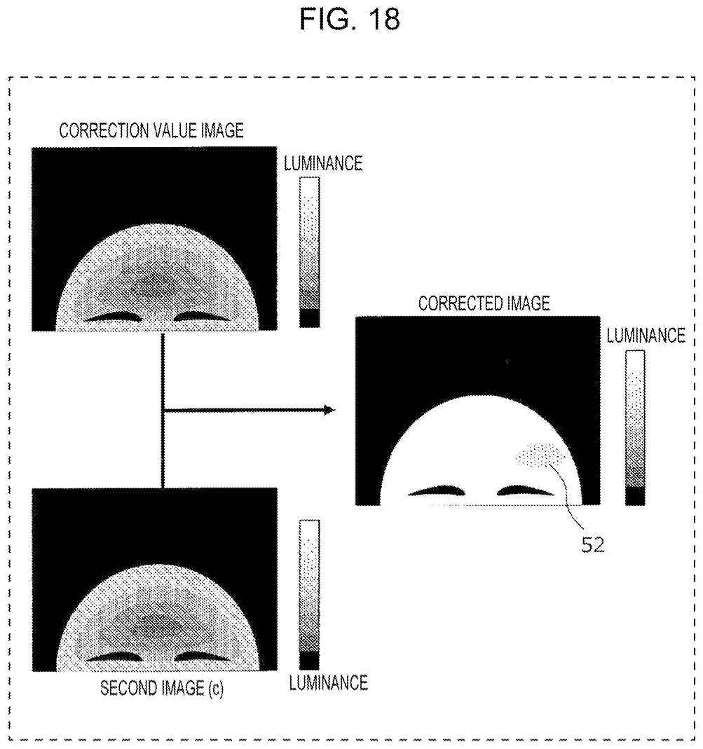

FIG. 18 is a diagram schematically illustrating processing for correcting the second image (c) using the correction value image. The signal processing circuit 134 corrects the second image (c) by dividing the value of each pixel of the second image (c) by the value of the corresponding pixel of the correction value image. Through this processing, the effect of changes in the luminance of the second image (c) due to the illuminance distribution of the light source 110 can be eliminated. Even in a case where body movement has occurred, the effect of the body movement is suppressed, and a region 52 where the cerebral blood flow changes can be clearly captured through measurement and arithmetic processing described above.

As described above, by acquiring different information from pixels in the same frame, the effect of changes in luminance due to the body movement of the subject 50 can be suppressed, and the state of the person's cerebral blood flow can be acquired with high temporal resolution and high accuracy.

Next, yet another modification of a measurement operation performed by the measurement apparatus 100 will be described.

FIG. 19 is a diagram illustrating a measurement operation according to another modification. FIG. 19 is a timing chart illustrating relationships between each exposure period of two charge accumulators of each of the first pixels P1 and the second pixels P2 and light emission pulses and reflected light pulses of two kinds having different wavelengths according to the present modification. In the present modification, similarly to as in the example illustrated in FIG. 4A, a first light pulse having a first wavelength greater than or equal to 650 nm and less than 805 nm and a second light pulse having a second wavelength greater than 805 nm and less than or equal to 950 nm are repeatedly emitted in an alternating manner in the periods of individual frames. The operation in a first frame and that in a second frame illustrated in FIG. 19 are repeated in an alternating manner at a predetermined frame rate. In this embodiment, the exposure periods of the two charge accumulators of each first pixel P1 in the first frame are different from those in the second frame. In the first frame, the first charge accumulator and the second charge accumulator of each first pixel P1 are each controlled so as to be exposed to light in exposure periods in which the front end portions of the reflected light pulses having the first wavelength and the second wavelength are detected. Each exposure period starts before the rising period of the reflected light pulse starts and ends immediately after the rising period ends. In contrast, in the second frame, the first charge accumulator and the second charge accumulator of each first pixel P1 are each controlled so as to be exposed to light in exposure periods in which the rear end portions of the reflected light pulses having the first wavelength and the second wavelength are detected. Each exposure period starts immediately before the trailing period of the reflected light pulse starts and ends during the trailing period or after the trailing period ends. In any one of the frames, the first charge accumulator and the second charge accumulator of each second pixel P2 are each controlled so as to be exposed to light in exposure periods in which the rear end portions of the reflected light pulses having the first wavelength and the second wavelength are detected. Each exposure period starts after the trailing period of the reflected light pulse starts and ends after the trailing period ends.

In the example illustrated in FIG. 19, a period during which a measurement operation for the first frame is performed is referred to as a “first measurement period”, and a period during which a measurement operation for the second frame is performed is referred to as a “second measurement period”. In the first measurement period, an exposure period of the first charge accumulator of each first pixel P1 is referred to as a “first exposure period”, and an exposure period of the second charge accumulator of each first pixel P1 is referred to as a “third exposure period”. In the second measurement period, an exposure period of the first charge accumulator of each first pixel P1 is referred to as a “fourth exposure period”, and an exposure period of the second charge accumulator of each first pixel P1 is referred to as a “fifth exposure period”. Note that, in each measurement period, each of the exposure periods of the individual charge accumulators of the second pixels is referred to as a “second exposure period”. The first exposure period starts when a first time t1 elapses from the time at which the first light pulse is emitted in the first measurement period. The third exposure period starts when a second time t2 elapses from the time at which the second light pulse is emitted in the first measurement period. The fourth exposure period starts when a third time t3 elapses from the time at which the first light pulse is emitted in the second measurement period. The fifth exposure period starts when a fourth time t4 elapses from the time at which the second light pulse is emitted in the second measurement period. The third time t3 differs from the first time t1, and the fourth time t4 differs from the second time t2. In the example illustrated in FIG. 19, the first time t1 is equal to the second time t2, and the third time t3 is equal to the fourth time t4. The first time t1 may differ from the second time t2, and the third time t3 may differ from the fourth time t4.

The signal processing circuit 134 performs a calculation based on Equation (1) described above on the basis of signals of the first charge accumulator of each first pixel acquired in the first exposure period and the fourth exposure period, signals of the second charge accumulator of each first pixel acquired in the third exposure period and the fifth exposure period, or both. Consequently, the signal processing circuit 134 can calculate the distance from the measurement apparatus 100 to a measurement point corresponding to the pixel. Moreover, the signal processing circuit 134 generates, on the basis of signals of the first charge accumulator and the second charge accumulator of each second pixel acquired in the second exposure period, cerebral blood information representing the state of the cerebral blood flow. The signal processing circuit 134 may correct the cerebral blood information on the basis of distance information by using the method described above.

By performing measurements at the exposure timings according to the present modification, the time difference between the acquisition timings of images having the individual wavelengths is reduced, and thus information corresponding to two wavelengths can be acquired with high temporal resolution. As a result, more subtle changes in the cerebral blood flow can be captured.

A measurement apparatus according to the present disclosure is effective in biosensing specifically targeted for persons since measurement can be performed with high temporal resolution in a contactless manner.

What is claimed is:

1. A measurement apparatus comprising:
a light source that emits a first light pulse toward a target;
a sensor including light detection cells including a first light detection cell and a second light detection cell; and
an electronic circuit that controls the light source and the sensor and processes a signal output from the sensor,
wherein the electronic circuit
causes the light source to emit a first light pulse,
causes the first light detection cell to detect a first reflected light pulse in a first exposure period and generate, based on the first reflected light pulse detected in the first exposure period, a first signal, the first reflected light pulse being generated based on the first light pulse and coming from the target, the first exposure period including at least part of a period from when an intensity of the first reflected light pulse starts increasing to when the intensity of the first reflected light pulse starts falling,
causes the second light detection cell to detect the first reflected light pulse in a second exposure period and generate, based on the first reflected light pulse detected in the second exposure period, a second signal, the second exposure period including at least part of a trailing period from when the intensity of the first reflected light pulse starts falling to when the intensity of the first reflected light pulse stops falling,
generates, based on the first signal, and outputs first data representing a state of a surface of the target, and
generates, based on the second signal, and outputs second data representing a state inside the target.

2. The measurement apparatus according to claim 1,
wherein the second exposure period starts after the trailing period of the first reflected light pulse starts.

3. The measurement apparatus according to claim 1,
wherein the first exposure period includes at least part of a rising period from when the intensity of the first reflected light pulse starts increasing to when the intensity of the first reflected light pulse stops increasing.

4. The measurement apparatus according to claim 1,
wherein the first exposure period ends before the trailing period starts.

5. The measurement apparatus according to claim 1, wherein
the light detection cells include first light detection cells including the first light detection cell and second light detection cells including the second light detection cell, and
the electronic circuit
causes each of the first light detection cells to detect the first reflected light pulse in the first exposure period and generate, based on the first reflected light pulse detected in the first exposure period, the first signal,
causes each of the second light detection cells to detect the first reflected light pulse in the second exposure period and generate, based on the first reflected light pulse detected in the second exposure period, the second signal,
generates, based on first signals including the first signal, and outputs the first data, the first signals being output from the respective first light detection cells, and
generates, based on second signals including the second signal, and outputs the second data, the second signals being output from the respective second light detection cells.

6. The measurement apparatus according to claim 5,
wherein the number of first light detection cells is smaller than the number of second light detection cells.

7. The measurement apparatus according to claim 5,
wherein the number of first light detection cells is larger than the number of second light detection cells.

8. The measurement apparatus according to claim 5, wherein
the sensor is an image sensor,
the light detection cells are arranged in a matrix, and
the electronic circuit
generates, as the first data, image data based on the first signals, and
generates, as the second data, image data based on the second signals.

9. The measurement apparatus according to claim 5, wherein
the light detection cells are arranged in a matrix, and
rows or columns formed by the first light detection cells and rows or columns formed by the second light detection cells are aligned in an alternating manner.

10. The measurement apparatus according to claim 5, wherein
the light detection cells are arranged in a matrix, and
the first light detection cells and the second light detection cells are arranged in a checkerboard pattern.

11. The measurement apparatus according to claim 5, wherein
the target includes a person's head, and
the first data represents an appearance of a face of the head.

12. The measurement apparatus according to claim 1, wherein
the target includes a person's head, and
the second data represents a cerebral blood flow state of the head.

13. The measurement apparatus according to claim 1, wherein
the light detection cells further include a third light detection cell, and
the electronic circuit causes the first light detection cell or the third light detection cell to detect the first reflected light pulse and generate a third signal in a third exposure period, the third exposure period being different from the first exposure period and the second exposure period.

14. The measurement apparatus according to claim 13,
wherein the electronic circuit
causes the first light detection cell to generate the third signal in the third exposure period, and
generates, based on the first signal and the third signal, and outputs data representing a distance from the sensor to the target.

15. The measurement apparatus according to claim 1, wherein
the light source further emits a second light pulse toward the target, and
the electronic circuit
causes the light source to emit the second light pulse after the electronic circuit causes the light source to emit the first light pulse,
causes the first light detection cell to detect a second reflected light pulse in a third exposure period and generate, based on the second reflected light pulse detected in the third exposure period, a third signal, the second reflected light being generated based on the second light pulse and coming from the target, the third exposure period including at least part of a trailing period from when an intensity of the second reflected light pulse starts falling to when the intensity of the second reflected light pulse stops falling,
causes the second light detection cell to detect the second reflected light pulse in a fourth exposure period and generate, based on the second reflected light pulse detected in the fourth exposure period, a fourth signal, the fourth exposure period being different from the third exposure period and including at least part of the trailing period of the second reflected light pulse, and
generates, based on the first signal and the third signal, and outputs data representing a distance from the sensor to the target.

16. The measurement apparatus according to claim 1, wherein
the target includes a person's head, and
the electronic circuit generates, based on the first signal and the second signal, and outputs data representing the person's psychological state or body condition.

17. The measurement apparatus according to claim 1, wherein
the light source further emits a second light pulse toward the target,
a wavelength of the second light pulse differs from a wavelength of the first light pulse,
the first exposure period starts when a first time elapses from a time at which emission of the first light pulse is started,
the electronic circuit
causes the light source to emit the first light pulse and the second light pulse in each of the first measurement period and the second measurement period,
causes the first light detection cell to detect the first reflected light pulse in the first exposure period included in the first measurement period and a second reflected light pulse generated based on the second light pulse and coming from the target in a third exposure period, which starts when a second time elapses from a time at which emission of the second light pulse is started in the first measurement period, and generate the first signal,
causes the first light detection cell to detect the first reflected light pulse in a fourth exposure period, which starts when a third time elapses from a time at which emission of the first light pulse is started in the second measurement period, and the second reflected light pulse in a fifth exposure period, which starts when a fourth time elapses from a time at which emission of the second light pulse is started in the second measurement period, and generate a third signal, and
generates, based on the first signal and the third signal, data representing a distance from the sensor to the target,
the third time differs from the first time, and
the fourth time differs from the second time.

18. A measurement apparatus comprising:
a light source that emits one or more light pulses toward a living body;
a sensor including light detection cells including a first light detection cell and a second light detection cell; and
an electronic circuit,
wherein the electronic circuit
causes the light source to emit the one or more light pulses,
causes the first light detection cell to detect a first component of one or more reflected light pulses and generate a first signal, the one or more reflected light pulses being generated based on the one or more light pulses and coming from the living body,
causes the second light detection cell to detect a second component of the one or more reflected light pulses and generate a second signal,
causes the first light detection cell to detect a third component of the one or more reflected light pulses and generate a third signal,
generates, based on the first signal and the third signal, first data representing a distance from the sensor to the living body, and
generates, based on the second signal, second data representing a blood flow state of the living body.

19. The measurement apparatus according to claim 18,
wherein the electronic circuit corrects, based on the first data, the second data.

20. The measurement apparatus according to claim 18,
wherein the electronic circuit corrects, based on data representing a spatial distribution of illuminance of the one or more light pulses and the first data, the second data.

21. A method for controlling a measurement apparatus including a light source and a sensor including light detection cells including a first light detection cell and a second light detection cell, the method comprising:
causing the light source to emit a light pulse;
causing the first light detection cell to detect a reflected light pulse in a first exposure period and generate, based on the reflected light pulse detected in the first exposure period, a first signal, the reflected light pulse being generated based on the light pulse and coming from a target, the first exposure period including at least part of a period from when an intensity of the reflected light pulse starts increasing to when the intensity of the reflected light pulse starts falling;
causing the second light detection cell to detect the reflected light pulse in a second exposure period and generate, based on the reflected light pulse detected in the second exposure period, a second signal, the second exposure period including at least part of a trailing period from when the intensity of the reflected light pulse starts falling to when the intensity of the reflected light pulse stops falling;
generating, based on the first signal, and outputting first data representing a state of a surface of the target; and
generating, based on the second signal, and outputting second data representing a state inside the target.

22. A method for controlling a measurement apparatus including a light source and a sensor including light detection cells including a first light detection cell and a second light detection cell, the method comprising:
causing the light source to emit one or more light pulses;
causing the first light detection cell to detect a first component of one or more reflected light pulses and generate a first signal, the one or more reflected light pulses being generated based on the one or more light pulses and coming from a living body;
causing the second light detection cell to detect a second component of the one or more reflected light pulses and generate a second signal;
causing the first light detection cell to detect a third component of the one or more reflected light pulses and generate a third signal;
generating, based on the first signal and the third signal, first data representing a distance from the sensor to the living body; and
generating, based on the second signal, second data representing a blood flow state of the living body.
